(19) United States
(12) Patent Application Publication
(10) Pub. No.: US 2025/0195263 A1
(43) Pub. Date: Jun. 19, 2025

(54) DEVICES, SYSTEMS AND METHODS FOR VAGINAL THERAPEUTICS

(71) Applicant: COSM Medical Corp., Toronto (CA)

(72) Inventors: Derek SHAM, Toronto (CA); Ali BORAZJANI, Toronto (CA); Aye N. SAN, Toronto (CA); Hamed KALAMI, Toronto (CA); Alexandra BLOKKER, Toronto (CA); John PHILLIPS, Toronto (CA); Golafsoun AMERI, Toronto (CA); Gaurab CHATTERJEE, Toronto (CA)

(21) Appl. No.: 18/852,453

(22) PCT Filed: Mar. 28, 2023
(86) PCT No.: PCT/CA2023/050411

§ 371 Date: Sep. 28, 2024

Related U.S. Application Data
(60) Provisional application No. 63/362,066, filed on Mar. 29, 2022.

(51) Int. Cl. A61F 6/12; A61B 5/00; A61F 2/00

(52) U.S. Cl. CPC A61F 6/12; A61B 5/4337; A61F 2/0009

(57) ABSTRACT

A range of surgical treatment options for pelvic organ prolapse (POP) and urinary incontinence (UI) as well as non-surgical treatments exist for POP and UI. However, the fitting of physical non-surgical devices such as Kegel assist devices and pessaries is by best guess or trial-and-error. The inventors have established methods, processes and devices to provide measurements of user specific anatomy and physiology in a reproducible manner with devices and systems that remove measurement artifacts, errors, bias etc. whilst providing the patient with an improved experience and the medical personnel with ergonomic, efficient, and easy to use systems. Further, new and improved designs, processes and methods for providing user specific physical non-surgical devices based upon such measurements are established that incorporate user specific anatomy and physiology, user lifestyle, and user experiences into the design and implementation of custom vaginal therapeutic devices.

### CROSS-REFERENCE TO RELATED APPLICATIONS

This patent application claims the benefit of priority from U.S. Provisional Patent Application 63/362,066 filed Mar. 29, 2022.

### FIELD OF THE INVENTION

The present invention relates to vaginal therapeutic devices and more specifically to vaginal therapeutic device geometries as well as methods and systems for patient measurement for designing and manufacturing patient specific vaginal therapeutic devices.

### BACKGROUND OF THE INVENTION

Today, a range of surgical treatment options for pelvic organ prolapse (POP) and urinary incontinence (UI) as well as non-surgical treatments exist for POP and UI. These non-surgical treatment options include Kegel exercises, Kegel assist devices, pessaries, core/floor strengthening exercises, biofeedback, electrical stimulation, hormone replacement therapy, tibial nerve stimulation and support garments. At present, physical non-surgical devices such as Kegel assist devices and pessaries are fitted by best guess, trial-and-error. Pessaries being prosthetic devices that can be inserted into the vagina to support its internal structure as a remedy for urinary incontinence, fecal incontinence, cervical incompetence and/or pelvic organ prolapse. Whilst the literature is replete with information explaining to medical personnel how to fit a pessary or users how to tell if their pessary fits correctly this potentially exacerbates the situation as there are at least 20 designs of pessary alone with each available in multiple sizes, in some instances 10 or more. Accordingly, the type of pessary and its size both need to be factored into addressing the correct device for treating POP in a female user.

As discussed by the inventors previously, see for example World Intellectual Property Office patent application 2019/051,579 or U.S. Provisional Patent Application 63/133,913, both entitled “Methods and Systems for Vaginal Therapeutic Device Fitting”, it would be beneficial to replace the current manual processes of sizing, fitting etc. as well as pessary design with a personal pelvic health characterization and provisioning approach that factors user specific anatomy and physiology, user life style, user experiences, automated assessments etc. into provisioning custom vaginal therapeutic devices.

Accordingly, the user specific anatomy and physiology should be obtained in a reproducible manner with devices and systems that remove measurement artifacts, errors, bias etc. whilst providing the patient with an improved experience and the medical personnel with ergonomic, efficient, and easy to use systems exploiting combinations of dedicated multi-patient measurement equipment with user specific consumable items for cleanliness etc.

Further, the designs of pessaries should accommodate their design and manufacture as a user specific therapeutic device as well as supporting enhanced usability and performance.

Other aspects and features of the present invention will become apparent to those ordinarily skilled in the art upon review of the following description of specific embodiments of the invention in conjunction with the accompanying figures.

### SUMMARY OF THE INVENTION

It is an object of the present invention to mitigate limitations within the prior art relating to vaginal therapeutic devices and more specifically to vaginal therapeutic device geometries as well as methods and systems for patient measurement for designing and manufacturing patient specific vaginal therapeutic devices.

In accordance with an embodiment of the invention there is provided a device comprising: an outer ring;
a hollow central shaft having a first distal end proximate the outer ring and a second distal end:
a plurality of ribs, each rib of the plurality of ribs having a first end at a predetermined position on the central shaft and a second distal end at a predetermined position on the outer ring;
a pull-tab having a first end attached to another predetermined position on the outer ring and a second distal end: wherein
the pull-tab extends from its first end through an opening in a sidewall of the hollow central shaft and through the second distal end of the hollow central shaft.

In accordance with an embodiment of the invention there is provided a device comprising:
a hollow body; and
a hollow central shaft having a first distal end connected to the hollow body at a predetermined position; and
a non-return valve coupled to a second distal end of the hollow central shaft: wherein the hollow body can be filled with a fluid once inserted into a vagina of a user of the device.

In accordance with an embodiment of the invention there is provided a device comprising:
a body providing a sheet of predetermined geometry and having an opening extending through the thickness of the sheet:
a hollow central shaft attached to a first side of the body such that a first end of the hollow central shaft is fluidically coupled to the opening within the body and a second distal end of the hollow central shaft is disposed away from the body:
a knob attached to the first side of the body at a predetermined position on the periphery of the body extended away from the body: wherein
the hollow central shaft has a predetermined geometry such that the second distal end of the hollow central shaft is offset at a predetermined angle from an axis perpendicular to the sheet at the first distal end of the hollow central shaft; and
the hollow central shaft has a predetermined geometry.

In accordance with an embodiment of the invention there is provided a vaginal therapeutic device (VTD) comprising:
an outer ring; and
a structure attached to the outer ring.

In accordance with an embodiment of the invention there is provided a device comprising:
a central body having a longitudinal axis;
a clinician arm having a first end disposed within the central body and a second distal end disposed external to the central body:
a first patient arm having a first end and a second distal end, where the first patient arm is disposed at a predetermined position with respect to the central body on a first side of the longitudinal axis, connected at a predetermined position to the central body via a first pivot, and connected via a first linkage mechanism to the clinician arm;
a second patient arm having a first end and a second distal end, where the second patient arm is disposed at a predetermined position with respect to the central body on a second side of the longitudinal axis, connected at a predetermined position to the central body via a second pivot, and connected via a second linkage mechanism to the clinician arm; wherein
movement of the clinician arm relative to the central body results in translation of the second distal ends of the first patient arm and the second patient arm laterally with respect to the longitudinal axis.

Other aspects and features of the present invention will become apparent to those of ordinarily skilled in the art upon review of the following description of specific embodiments of the invention in conjunction with the accompanying figures.

### BRIEF DESCRIPTION OF THE DRAWINGS

Embodiments of the present invention will now be described, by way of example only, with reference to the attached Figures, wherein:

FIG. 1 depicts an exemplary process flow for providing a user with a user specific therapeutic device (USTD) according to an embodiment of the invention;

FIG. 2 depicts an exemplary USTD platform and system supporting embodiments of the invention:

FIGS. 3A to 3C depict physical geometry design parameters and exemplary design graphical user interface (GUI) for specifying a ring-type USTD according to embodiments of the invention:

FIGS. 3D and 3E depict a ring-type USTD with pull-tab according to embodiments of the invention:

FIGS. 4A to 4E depict physical geometry design parameters and an exemplary design GUI for specifying a Gellhorn-type USTD according to embodiments of the invention:

FIGS. 5A to 5C depict physical geometry design parameters for a Marland type USTD according to embodiments of the invention:

FIGS. 6A and 6B depict a Gellhorn-type USTD according to an embodiment of the invention with a first variant of a pull tab according to an embodiment of the invention:

FIGS. 7A and 7B depict a Gellhorn-type USTD according to an embodiment of the invention with a second variant of a pull tab according to an embodiment of the invention:

FIG. 7C depicts a USTD Gellhorn style with pull-tab according to embodiments of the invention:

FIG. 8 depicts cap folding force of commercial Gellhorn pessaries against user specific USTD Gellhorn devices according to embodiments of the invention:

FIG. 9 depicts maximum bending force of commercial Gellhorn pessaries against user specific USTD Gellhorn devices according to embodiments of the invention:

FIG. 10 depicts stem insertion versus pull tab testing of commercial Gellhorn pessaries against user specific USTD Gellhorn devices according to embodiments of the invention;

FIG. 11 depicts an inflatable Gellhorn type USTD according to an embodiment of the invention;

FIG. 12 depicts a Gellhorn type USTD with knob for urinary incontinence (UI) according to an embodiment of the invention:

FIG. 13 depicts a collapsible pyramid type USTD according to an embodiment of the invention;

FIGS. 14A-14D respectively depict ring-type USTDs according to embodiments of the invention with or without support and with or without knob configuration with a first pull tab variant:

FIGS. 15A and 15B depict ring-type USTDs according to embodiments of the invention with second and third pull tab variants:

FIGS. 16A and 16B depict Marland-type USTDs according to embodiments of the invention with second and third pull tab variants:

FIGS. 17A and 17B depict V-shaped double ring-type USTDs according to embodiments of the invention with a pull tab;

FIGS. 18A to 18C depict a POP-Q+ measurement tool according to an embodiment of the invention;

FIG. 19 depicts a POP-Q+ measurement tool according to an embodiment of the invention;

FIGS. 20A and 20B depict a POP-Q+ measurement tool according to an embodiment of the invention:

FIGS. 20C and 20D depict a POP-Q+ measurement tool according to an embodiment of the invention

FIG. 21 depicts a non-linear scale for a POP-Q+ measurement tool according to an embodiment of the invention:

FIG. 22 depicts a conical scale for a POP-Q+ measurement tool according to an embodiment of the invention;

FIG. 23 depicts a POP-Q+ measurement tool according to an embodiment of the invention;

FIGS. 24A and 24B depict POP-Q+ measurement tools according to embodiments of the invention;

FIG. 25 depicts non-perforated cube pessary according to the prior art and embodiments of the invention:

FIG. 26 depicts perspective views and dimensions for perforated cube pessaries according to embodiments of the invention;

FIGS. 27 to 29 depict POP-Q+ measurement tools according to embodiments of the invention;

FIG. 30 depicts a driving mechanisms for fluidic control of a POP-Q+ measurement tool according to an embodiment of the invention:

FIG. 31 depicts an alternate mechanical driving mechanism for a POP-Q+ measurement tool according to an embodiment of the invention; and

FIG. 32 depicts a glove based POP-Q+ measurement tool for augmenting a manual finger method.

### DETAILED DESCRIPTION

The present invention is directed to vaginal therapeutic devices and more specifically to vaginal therapeutic device geometries as well as methods and systems for patient measurement for designing and manufacturing patient specific vaginal therapeutic devices.

The ensuing description provides representative embodiment(s) only, and is not intended to limit the scope, applicability, or configuration of the disclosure. Rather, the ensuing description of the embodiment(s) will provide those skilled in the art with an enabling description for implementing an embodiment or embodiments of the invention. It being understood that various changes can be made in the function and arrangement of elements without departing from the spirit and scope as set forth in the appended claims. Accordingly, an embodiment is an example or implementation of the inventions and not the sole implementation. Various appearances of “one embodiment,” “an embodiment” or “some embodiments” do not necessarily all refer to the same embodiments. Although various features of the invention may be described in the context of a single embodiment, the features may also be provided separately or in any suitable combination. Conversely, although the invention may be described herein in the context of separate embodiments for clarity, the invention can also be implemented in a single embodiment or any combination of embodiments.

Reference in the specification to “one embodiment,” “an embodiment,” “some embodiments” or “other embodiments” means that a particular feature, structure, or characteristic described in connection with the embodiments is included in at least one embodiment, but not necessarily all embodiments, of the inventions. The phraseology and terminology employed herein is not to be construed as limiting but is for descriptive purpose only. It is to be understood that where the claims or specification refer to “a” or “an” element, such reference is not to be construed as there being only one of that element. It is to be understood that where the specification states that a component feature, structure, or characteristic “may,” “might,” “can” or “could” be included, that particular component, feature, structure, or characteristic is not required to be included.

Reference to terms such as “left,” “right,” “top,” “bottom,” “front” and “back” are intended for use in respect to the orientation of the particular feature, structure, or element within the figures depicting embodiments of the invention. It would be evident that such directional terminology with respect to the actual use of a device has no specific meaning as the device can be employed in a multiplicity of orientations by the user or users. Reference to terms “including.” “comprising.” “consisting” and grammatical variants thereof do not preclude the addition of one or more components, features, steps, integers, or groups thereof and that the terms are not to be construed as specifying components, features, steps, or integers. Likewise, the phrase “consisting essentially of,” and grammatical variants thereof, when used herein is not to be construed as excluding additional components, steps, features integers or groups thereof but rather that the additional features, integers, steps, components, or groups thereof do not materially alter the basic and novel characteristics of the claimed composition, device, or method. If the specification or claims refer to “an additional” element, that does not preclude there being more than one of the additional element.

“Artificial intelligence” (AI, also machine intelligence, MI) as used herein may refer to, but is not limited to, intelligence exhibited by machines rather than humans or other animals which exhibit so-called natural intelligence (NI). Colloquially, the term AI is employed when a machine mimics “cognitive” functions which humans associate with other human minds, such as “learning” and “problem solving”. AI may employ one or more tools, including, but not limited to search and optimization, logic, probabilistic methods for uncertain reasoning, classifiers and statistical learning methods, neural networks, deep feedforward neural networks, deep recurrent neural networks, and control theory.

A “portable electronic device” (PED) as used herein and throughout this disclosure, refers to a wireless device used for communications and other applications that requires a battery or other independent form of energy for power. This includes devices, but is not limited to, such as a cellular telephone, smartphone, personal digital assistant (PDA), portable computer, pager, portable multimedia player, portable gaming console, laptop computer, tablet computer, a wearable device, an electronic reader, a vaginal therapy device (VTD), and a user specific therapeutic device (USTD).

A “fixed electronic device” (FED) as used herein and throughout this disclosure, refers to a wireless and/or wired device used for communications and other applications that requires connection to a fixed interface to obtain power. This includes, but is not limited to, a laptop computer, a personal computer, a computer server, a kiosk, a gaming console, a digital set-top box, an analog set-top box, an Internet enabled appliance, an Internet enabled television, and a multimedia player.

An “application” (commonly referred to as an “app”) as used herein may refer to, but is not limited to, a “software application,” an element of a “software suite,” a computer program designed to allow an individual to perform an activity, a computer program designed to allow an electronic device to perform an activity, and a computer program designed to communicate with local and/or remote electronic devices. An application thus differs from an operating system (which runs a computer), a utility (which performs maintenance or general-purpose chores), and a programming tools (with which computer programs are created). Generally, within the following description with respect to embodiments of the invention an application is generally presented in respect of software permanently and/or temporarily installed upon a PED and/or FED.

A “user” as used herein may refer to, but is not limited to, an individual exploiting a vaginal therapeutic device according to an embodiment or embodiments of the invention. As such an individual may be employing a vaginal therapeutic device with respect to one or more conditions, requirements, and/or preventions. As such an individual may include, but not be limited to, a person with a vagina, an animal with a vagina, a recipient of gender affirming surgery (also known as sex reassignment surgery, gender confirmation surgery, gender specific reconstruction surgery, and sex realignment surgery). In its broadest sense the user may further include, but not be limited to, mechanical systems, robotic systems, android systems, etc. that may be characterised by a requirement to exploit one or more embodiments of the invention. A user may be associated with biometric data which may be, but not limited to, monitored, acquired, stored, transmitted, processed and analysed either locally or remotely to the user. A user may also be associated through one or more accounts and/or profiles with one or more of a service provider, third party provider, enterprise, social network, social media etc. via a dashboard, web service, website, software plug-in, software application, and graphical user interface.

The terms “woman” or “female” as used herein, and throughout this disclosure, refers to a human having a vagina or surgically formed vaginal structure and optionally a clitoris or clitoral region, uterus, bladder, a urethra, rectum, and/or an anus. The terms “woman” and “female” are used interchangeably herein.

“User information” as used herein may refer to, but is not limited to, user behavior information and/or user profile information. It may also include a user's biometric information, an estimation of the user's biometric information, or a projection/prediction of a user's biometric information derived from current and/or historical biometric information.

A “vaginal therapeutic device” (VTD, commonly referred to as a pessary) refers to a medical device and is a specific form of a user specific therapeutic device (USTD). A USTD may be used to support the uterus, vagina, bladder, or rectum. A USTD may be employed to treat a pelvic organ prolapse (POP), such as prolapse of the uterus for example, treat an intestinal issue, an enterocele (essentially a vaginal hernia), reduce the impact of an evolving POP, treat and/or reduce the impact of urinary incontinence (UI), treat and/or reduce the impact of stress UI, and treat and/or reduce the impact of urge UI. Alternatively, a USTD may be employed during pregnancy to treat an incompetent (or insufficient) cervix (cervix starts to shorten and open too early) as an alternative to cervical cerclage since there are fewer potential complications. A USTD may also be used to address constipation, fecal incontinence, retroverted uterus, address cystocele, address rectocele, manage menstruation, induce an abortion, or provide and/or support contraception. A USTD may be placed temporarily or permanently. A pharmaceutical USTD may provide an effective means for the delivery of one or more pharmaceutical substances which are easily absorbed through the mucosa of the vagina, or intended to have action in the locality, for example to delivery hormones or act against inflammation or infection, or on the uterus. An occlusive USTD may perform similarly to a cervical cap and may be used in combination with spermicide as a contraception. A stem USTD, a type of occlusive USTD, is an early form of the cervical cap shaped like a dome to cover the cervix but with a central rod or “stem” entering the os to hold it in place. USTD's within the prior art are offered in a variety of forms including, but not limited, ring USTDs, lever USTDs, Gehrung USTDs, inflatable USTDs, doughnut USTDs, cube USTDs, Gellhorn USTDs, and incontinence USTDs. USTDs according to embodiments of the invention are designed in dependence upon the user for custom fitting and/or applications including, but not limited to, prolapse, urinal incontinence, and fecal incontinence.

“Gender affirming surgery” (also known as gender reassignment surgery, gender confirmation surgery, genital reconstruction surgery, gender-affirming surgery, or gender realignment surgery) as used herein may refer to, but is not limited to, one or more surgical procedures that adjust a user's physical appearance and function with respect to their genitalia which may require the user to use a vaginal therapeutic device according to an embodiment of the invention.

A “wearable device” or “wearable sensor” relates to miniature electronic devices that are worn by the user including those under, within, with or on top of clothing and are part of a broader general class of wearable technology which includes “wearable computers” which in contrast are directed to general or special purpose information technologies and media development. Such wearable devices and/or wearable sensors may include, but not be limited to, smartphones, smart watches, e-textiles, smart shirts, activity trackers, smart glasses, environmental sensors, medical sensors, biological sensors, physiological sensors, chemical sensors, ambient environment sensors, position sensors, neurological sensors, drug delivery systems, medical testing and diagnosis devices, and motion sensors. The wearable devices and/or wearable sensors may include, but not be limited to, devices that can stimulate and/or measure parameters related to the function of the vagina, urethra, uterus, bladder, cervix, rectum, anal sphincter, urethral sphincter, and abdominal cavity. It may also be used to measure intra-abdominal pressure which can be correlated to the amount of force that the USTD will need to support.

“Biometric” information as used herein may refer to, but is not limited to, data relating to a user characterised by data relating to a subset of conditions including, but not limited to, their environment, medical condition, biological condition, physiological condition, chemical condition, ambient environment condition, position condition, neurological condition, drug condition, and one or more specific aspects of one or more of these said conditions. Accordingly, such biometric information may include, but not be limited, blood oxygenation, blood pressure, blood flow rate, heart rate, temperate, fluidic pH, viscosity, particulate content, solids content, altitude, vibration, motion, perspiration, EEG, ECG, energy level, etc. In addition, biometric information may include data relating to physiological characteristics related to the shape and/or condition of the body wherein examples may include, but are not limited to, fingerprint, facial geometry, baldness, DNA, hand geometry, odour, and scent. Biometric information may also include data relating to behavioral characteristics, including but not limited to, typing rhythm, gait, and voice.

A “profile” as used herein, and throughout this disclosure, refers to a computer and/or microprocessor readable data file comprising data relating to a USTD according to an embodiment of the invention and/or biometric data of a user.

A “scaffold” or “scaffolds” as used herein, and throughout this disclosure, refers to a structure that is used to hold up, interface with, or support another material or element(s). This includes, but is not limited to, such two-dimensional (2D) structures such as substrates and films, three-dimensional (3D) structures such as geometrical objects, non-geometrical objects, combinations of geometrical and non-geometrical objects, naturally occurring structural configurations, and manmade structural configurations. A scaffold may be solid, hollow, and porous or a combination thereof. A scaffold may contain recesses, pores, openings, holes, vias, and channels or a combination thereof. A scaffold may be smooth, textured, have predetermined surface profiles and/or features. A scaffold may be intended to support one or more other materials, one or more films, a multilayer film, one type of particle, multiple types of particles etc. A scaffold may include, but not be limited to, a spine of a device and/or a framework, for example, which also supports a shell and/or a casing.

A “shell” as used herein, and throughout this disclosure, refers to a structure that is used to contain and/or surround at least partially and/or fully a number of elements within devices according to embodiments of the invention. A shell may include, but not limited to, a part or parts that are mounted to, attached to, and/or surround all or part of a scaffold or scaffolds that support elements within a device according to an embodiment of the invention.

A “casing” or “skin” as used herein, and throughout this disclosure, refers to a structure surrounding a scaffold and/or shell. This includes structures typically formed from an elastomer and/or silicone to provide a desired combination of physical tactile surface properties to the device it forms part of and other properties including, but not limited to, hermeticity, liquid ingress barrier, solid particulate ingress barrier, surface sheen, and colour. A casing may include, but not limited to, a part or parts that are mounted to a scaffold or scaffolds and/or a casing or casings forming part of a device according to an embodiment of the invention.

A “resin” as used herein may refer to, but is not limited to, a solid or highly viscous substance which is typically convertible into polymers. Resins may be plant-derived or synthetic in origin.

A “polymer” as used herein may refer to, but is not limited to, is a large molecule, or macromolecule, composed of many repeated subunits. Such polymers may be natural and synthetic and typically created via polymerization of multiple monomers. Polymers through their large molecular mass may provide unique physical properties, including toughness, viscoelasticity, and a tendency to form glasses and semi-crystalline structures rather than crystals.

A “polyester” as used herein, and throughout this disclosure, refers to a category of polymers that contain the ester functional group in their main chain. This includes but is not limited to polyesters which are naturally occurring chemicals as well as synthetics through step-growth polymerization, for example. Polyesters may be biodegradable or not. Polyesters may be a thermoplastic or thermoset or resins cured by hardeners. Polyesters may be aliphatic, semi-aromatic or aromatic. Polyesters may include, but not be limited to, those exploiting polyglycolide, polylactic acid (PLA), polycaprolactone (PCL), polyhydroxyalkanoate (PHA), polyhydroxybutyrate (PHB), polyethylene adipate (PEA), polybutylene succinate (PBS), polyethylene terephthalate (PET), polybutylene terephthalate (PBT), polytrimethylene terephthalate (PTT), and polyethylene naphthalate (PEN).

A “thermoplastic” or “thermosoftening plastic” as used herein and throughout this disclosure, refers to a category of polymers that become pliable or moldable above a specific temperature and solidify upon cooling. Thermoplastics may include, but not be limited, polycarbonate (PC), polyether sulfone (PES), polyether ether ketone (PEEK), polyethylene (PE), polypropylene (PP), poly vinyl chloride (PVC), polytetrafluoroethylene (PTFE), polyimide (PI), polyphenylsulfone (PPSU), polychlorotrifluoroethene (PCTFE or PTFCE), fluorinated ethylene propylene (FEP), and perfluoro alkoxy alkane (PFA).

An “aramid” as used herein, and throughout this disclosure, refers to an aromatic polyamide. Aramids are a class of materials fibers in which the chain molecules are highly oriented along the fiber axis, so the strength of the chemical bond can be exploited. Examples include but are not limited to fibers distributed under brand names such as Kevlar™, Technora™, Twaron™, Heracron™, Nomex™, Innegra S™ and Vectran™ as well as nylon and ultra-high molecular weight polyethylene.

A “silicone” as used herein, and throughout this disclosure, refers to a polymer that includes any inert, synthetic compound made up of repeating units of siloxane.

An “elastomeric” material or “elastomer” as used herein, and throughout this disclosure, refers to a material, generally a polymer, with viscoelasticity. Elastomers may include, but not be limited to, unsaturated rubbers such as polyisoprene, butyl rubber, ethylene propylene rubber, silicone rubber, fluorosilicone rubber, fluoroelastomers, perfluoroelastomers, and thermoplastic elastomers.

The term “flexible,” as used herein, refers to the ability of a body that is capable of being bent or flexed and refers to the ability of a body that has been subjected to an external force to return to its original size and/or shape once the external force has been removed or reduced to below a particular level. Something that is flexible can be, for example, resilient or malleable. A “flexible” material, such as a rubber for example, may be characterised by a low Young's modulus.

The term “resilient,” as used herein, refers to the ability of a body that has been subjected to an external force to recover, or substantially recover, its original size and/or shape, following deformation. The term “malleable,” as used herein, refers to the ability of a body that has been subjected to an external force to deform and maintain, or substantially maintain, the deformed size and/or shape. Accordingly, a malleable material supports plastic deformation. A resilient material, such as polytetrafluorethylene for example, may be characterised by a moderate Young's modulus. A rigid material, for example steel, may be characterised by a high Young's modulus but may under appropriate conditions undergo plastic deformation.

A “CAD model” as used herein may refer to, but is not limited to, an electronic file containing information relating to a component, piece-part, element, assembly to be manufactured. A CAD model may define an object within a two-dimensional (2D) space or a three-dimensional (3D) space and may in addition to defining the internal and/or external geometry and structure of the object include information relating to the material(s), process(es), dimensions, tolerances, etc. Within embodiments of the invention the CAD model may be generated and transmitted as electronic content to a system providing manufacturing according to one or more embodiments of the invention. Within other embodiments of the invention the CAD model may be derived based upon one or more items of electronic content directly, e.g. a 3D model may be created from a series of 2D images or extracted from electronic content.

A “fluid” as used herein may refer to, but is not limited to, a substance that continually deforms (flows) under an applied shear stress. Fluids may include, but are not limited to, liquids, gases, plasmas, and some plastic solids.

A “powder” as used herein may refer to, but is not limited to, a dry, bulk solid composed of a large number of very fine particles that may flow freely when shaken or tilted. Powders may be defined by both a combination of the material or materials they are formed from and the particle dimensions such as minimum, maximum, distribution etc. A powder may typically refer to those granular materials that have fine grain sizes but may also include larger grain sizes depending upon the dimensions of the part being manufactured, the characteristics of the additive manufacturing system etc.

“Additive manufacturing” (AM) as used herein may refer to, but is not limited to, a process or processes used to create a three-dimensional object in which layers of material are formed under computer control. Commonly referred to as “3D printing” the processes of AM are currently defined in ISO/ASTM52900-15, which defines several categories of AM processes although others may also be viewed as AM processes. These categories being binder jetting, directed energy deposition, material extrusion, material jetting, powder bed fusion, sheet lamination and vat photopolymerization. “3D printers” exploiting custom “inkjet” print heads are a special application of plastic extrusion known as fused deposition modelling. AM processes may be applied to plastics, ceramics, and metals. AM processes for AM sintering or melting include selective laser sintering, direct metal laser sintering, and selective laser melting whilst those for deposition may include microcasting and sprayed materials. In some instances, sacrificial and/or support materials may be employed in conjunction with AM processes to achieve the desired geometry and/or combination of materials.

“Non-additive manufacturing” (NAM) as used herein may refer to, but is not limited to, a process or processes used to create a three-dimensional object by subtractive or transformative manufacturing. NAM processes may include, but not be limited to, hydroforming, stamping, injection molding, casting, machining, and welding.

As noted above a range of surgical treatment options for POP as well as non-surgical treatments exist. Non-surgical treatment options include Kegel exercises. Kegel assist devices, pessaries, core/floor strengthening exercises, biofeedback, and support garments. Electrical stimulation, hormone replacement therapy and tibial nerve stimulation provide non-surgical treatment options to other pelvic floor disorders (PFDs). At present, physical non-surgical devices such as pessaries are fitted today by best guess, trial-and-error. Whilst the literature is replete with information explaining to medical personnel how to fit a pessary or users how to tell if their pessary fits correctly this potentially exacerbates the situation as there are at least 20 designs of pessary alone each available in multiple sizes, in some instances 10 or more. So, type of pessary and size both need to be factored into addressing the correct device for a user. As discussed by the inventors previously, see for example World Intellectual Property Office patent application 2019/051,579 or U.S. Provisional Patent Application 63/133,913, both entitled “Methods and Systems for Vaginal Therapeutic Device Fitting”, it would be beneficial to replace the current manual processes of sizing, fitting etc. as well as pessary design with a personal pelvic health characterization and provisioning approach that factors user specific anatomy and physiology, user life style, user experiences, automated assessments etc. into provisioning custom vaginal therapeutic devices.

Accordingly, the inventors have established custom USTD processes wherein core advantages include simplifying the fitting process as well as establishing a new paradigm between the two characteristics of support and comfort which runs counter to prior art USTDs the more support the less comfortable, less prone to expulsion during exercise, or tissue erosion over long term. Accordingly, the process established by the inventors resets this paradigm through a custom fitting and manufacturing process with a single material or multiple material USTD design allowing support to be established from a scaffold within the device whilst a shell and/or skin around the scaffold provide for increased comfort. Further, adoption of additive manufacturing processes allows the custom USTD manufacturing to be established in multiple locations with a city, state, province, country allowing improved delivery, responsiveness and supporting exploitation of custom USTDs with reduced usage duration as they exploit anti-microbial coatings, contraceptive coating, etc.

Accordingly, the user specific anatomy and physiology should be obtained in a reproducible manner with devices and systems that remove measurement artifacts, errors, bias etc. whilst providing the patient with an improved experience and the medical personnel with ergonomic, efficient, and easy to use systems exploiting combinations of dedicated multi-patient measurement equipment with user specific consumable items for cleanliness etc.

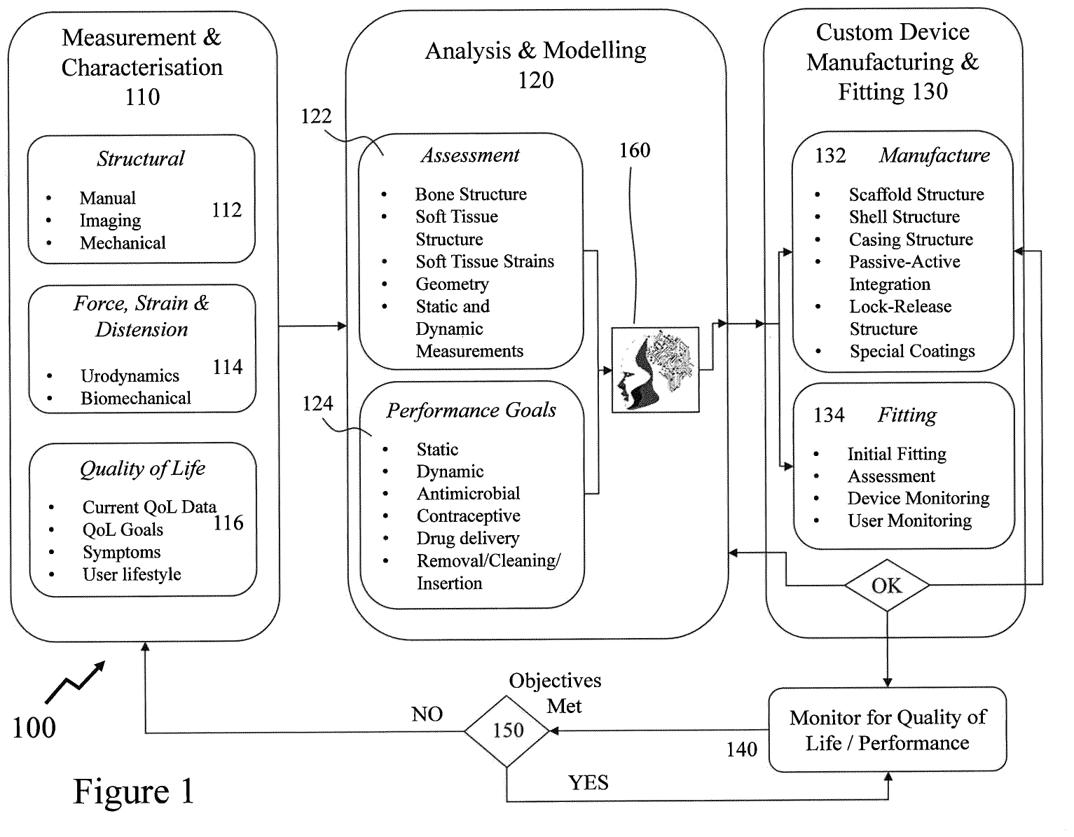

Accordingly, referring to FIG. 1 there is depicted an exemplary process Flow 100 for providing a user with a custom USTD according to an embodiment of the invention such that the process is reduced from a bewildering array of USTD types and dimensions to a single USTD option without significant effort from either the patient or the clinician. Accordingly, at step 110 the process begins with the step of Measurement and Characterisation (M&C) 110 before progressing to Analysis and Modelling (A&M) 120 and Custom Device Manufacturing and Fitting (CUDEMAF) 130 wherein the patient (user) is now provided and fitted with a custom USTD. Next, the process proceeds to step 140 wherein ongoing monitoring of quality of life (QOL) and performance of the USTD wherein a decision process 150 may determine whether the objectives of the USTD are being met or still being met on an ongoing basis and hence determine whether monitoring should continue or whether the process should begin again with step 110.

An ongoing monitoring and cyclic process may be appropriate for a variety of USTD use cases including, but not limited to:
changing physical characteristics of the user as they get older which may be more gradual in older users such as adults or the elderly and more rapid in younger users:
changing physiology of the user wherein additional symptoms and/or conditions manifest themselves:
changing physiology of the user in that muscles and tissue resilience, strength, compliance etc. may change; and degradation in the USTD itself.

Accordingly, as depicted M&C 110 comprises three sub-processes, these being:
Structural 112;
Force, Strain and Distension 114; and
Quality of Life 116.

Within embodiments of the invention the custom USTD may be employed in combination with other therapies and/or pharmaceutical coatings etc. in order to combine a custom USTD with regenerative medicine. Accordingly, within other embodiments of the invention a USTD according to an embodiment of the invention may exploit an energy delivery system such as electrical stimulation, infrared irradiation or ultraviolet irradiation for example. A custom USTD may also be employed in conjunction with other medical procedures and/or treatment regimens including, for example, exploitation of biological therapies including recombinant proteins, recombinant peptides, and stem cells for example.

Structural 112 may comprise one or more measurements of the user's anatomy and/or measurements of the user's physical characteristics such that one or more characteristics such as the dimensions of the user's major anatomical structures, anatomical geometry, etc. are defined. For example, a Pelvic Organs Prolapse Quantification (POP-Q) may be performed, this being a standardized tool for documenting the examination findings recognized by International Continence Society (ICS) and International Urogynecological Association (IUGA). Within the POP-Q system six principal landmarks are defined to describe the degree (quantity) of Pelvic Organ Prolapse (POP). These points (Aa, Ba, C, D. Ap, Bp) are located on vaginal walls and cervix and are related to the hymen which is considered a fixed point of reference. Another three distances (GH, TVL, and PB) may also be defined for more detailed description. The “stage” of prolapse is typically defined according to the evaluation of these points. These nine points are defined by letters Aa, Ba, C, D, Ap, Bp, GH, TVL, and PB respectively, these being:
Point Aa: This point is located in the midline anterior vaginal wall approximately 3 cm proximal from the external urethral meatus. The range of its position relative to hymen is typically from −3 cm to +3 cm.
Point Ba: The most distal position of any part in the anterior vaginal wall from the vaginal cuff or anterior vaginal fornix to Point Aa. In absence of prolapse, this point is at −3 cm. Point C: The most distal edge of the cervix or vaginal cuff (hysterectomy scar) after total hysterectomy.
Point D: Represents the pouch of Douglas or the location of posterior vaginal fornix. It is also a point of measurement for differentiation apical compartment prolapse from cervical elongation. Accordingly, in the absence of a cervix point D is omitted.
Point Ap: Located in the middle of posterior vaginal wall 3 cm proximal to the hymen. The range of its position relative to hymen is typically from −3 cm to +3 cm.
Point Bp: Represents the most distal position of any part in the posterior vaginal wall from the vaginal cuff or posterior vaginal fornix to point Ap.
Genital hiatus (GH): The distance between external urethral meatus and posterior margin of the hymen.
Total Vaginal Length (TVL): The deepest length of the vagina (cm) measured when point D (or the vaginal cuff) are reduced to normal position.
Perineal Body (PB): The distance measured from posterior margin of the hymen to the mid-anal opening.

All measurements are taken on Valsalva except TVL. A clinician may employ a manual procedure to measure the basic six or full nine points Aa, Ba, C, D, Ap, Bp, GH, TVL, and PB, respectively. This may be via the use of a ruler, swab, or other mechanical measuring device. The necessary user-specific structural/anatomical parameters may also be derived from one or more imaging techniques including, but not limited to, ultrasound imaging, magnetic resonance imaging (MRI), elastography, acoustic analysis, tactile imaging, photoacoustic (optoacoustic) imaging, tomography, echocardiography, functional near-infrared spectroscopy, and electrical impedance tomography. Alternatively, mechanical based devices may be employed to perform measurements and/or support one or more transducers for one or more imaging techniques, manual processes etc. Further these measurements may be at least one intravaginal, translabial, and transperineal.

For example, within an embodiment of the invention, ultrasound imaging may be used to determine specific anatomic parameters such as cross-sectional diameter of the vagina at various cross-sections along its length. Distances between various anatomical structures may also be used to determine specific anatomical parameters including but not limited to distances between any of the following anatomical structures: pubic symphysis, cervix (anterior lip, posterior lip, or os), urethra, bladder neck, bladder, rectum, anus, or levator ani and other pelvic floor musculature. Importantly, mobility of the various anatomical structures may also be measured by obtaining measurements at rest and on maximum Valsalva and contraction. These mobility measures help characterize the prolapsing compartment(s) and have been correlated with patient's symptoms. For example, bladder descent greater than 1 cm below the pubic symphysis on Valsalva is correlated with symptoms of prolapse. In one embodiment, these data may be used to generate a prosthetic that optimally fits within the vagina and limits bladder descent and thereby minimizes patients' symptoms. In addition to distances, thicknesses, surface area, and shape of the vaginal wall may also be assessed using ultrasound. Thickness data may be used to customize the mechanical properties and shape of the prosthetic device such that it minimizes the risk for vaginal ulcerations and erosions.

Force, Strain and Distension 114 may comprise one or more measurements of characteristics of the user's anatomy and/or measurements of the user's physical characteristics such as compliance/resilience of the user's tissues, the movement(s) and strength of user's musculature within the appropriate anatomical regions. These may involve mechanical and/or imaging testing discretely or in combination with other tests. Such tests may include, but not be limited to:
Vaginal manometry.
Vaginal distension with imaging from any imaging modality such as, but not limited to, ultrasound, magnetic resonance imaging (MRI), endoscope, LIDAR and X-ray for example.
Urodynamic measurements including, but not limited to:
   Post-void residual volume via insertion of a urinary catheter or with the aid of a bladder scanner:
   Uroflowmetry where “free” uroflowmetry measures the rate of bladder evacuation, “pressure” uroflowmetry combines rate of voiding measurements with simultaneous assessment of bladder and rectal pressures;
   Multichannel cystometry which exploits a pair of pressure monitoring catheters to measure the pressure in the rectum and in the bladder to deduce the presence of contractions of the bladder wall, during bladder filling, or during other provocative maneuvers. The strength of the urethra can also be tested during the filling phase, using a cough or Valsalva maneuver, to confirm genuine stress incontinence; and
   Urethral pressure profilometry which measures the strength of sphincter contraction.
Tactile imaging for force and strain measurements.
Elastography from ultrasound or MRI as well as other intravaginal measurements and perineal measurements.
Electromyography (EMG) measurements of electrical activity of the pelvic floor muscles.
Fluoroscopy, dynamic X-ray sequences, of the bladder and bladder neck during voiding.
Intravaginal molding or casts.
Finger palpation

Techniques may include those identified supra and others including, but not be limited, leak point pressure, vaginal manometry, ultrasound, elastography, strain sensor array, acoustic analysis, tactile imaging, and photoacoustic (optoacoustic) imaging. The measurements performed within Structural 112 and Force, Strain and Distension 114 may be statically acquired, i.e. with the user sitting/laying/standing within a clinic or another environment and/or dynamically acquired with the user performing one or more routine aspects of their life such as Valsalva effort, walking, exercising, running, lifting, bending, etc.

In contrast to the Structural 112 and Force, Strain and Distension 114 the Quality of Life 116 is user reported assessment. Accordingly, Quality of Life (QOL) 116 may include, but not limited to:
Current QOL data for the user (patient) using validated questionnaires such as the Pelvic Floor Distress Inventory (PFDI) and Pelvic Floor Impact Questionnaire (PFIQ) for example:
QOL goals for the user (patient);
Symptoms experienced by the user; and
User lifestyle.

Accordingly, QOL 116 establishes baseline QOL data which may be employed subsequently for the monitoring, QOL and performance of the USTD once manufactured and employed according to embodiments of the invention. Accordingly, for one user a QoL goal may be the elimination of a symptom that occurs only during sexual activity whilst for another it may be during a specific exercise, sporting activity, etc. or for another over specific periods of time and/or generally monitored etc. Additionally, the USTD in terms of being permanent, semi-permanent, or temporary is established wherein for temporary use at least the installation/removal means and/or mechanisms are established with the user. For permanent and semi-permanent the installation/removal means are geared primarily to the clinician rather than the user.

In establishing the QoL 116 a user may employ an application upon a PED and/or FED in order to track the user's (patient's) perceived QoL, to monitor and/or log occurrences such as incontinence, pain, prolapse symptoms, pessary fall out, etc.

From M&C 110 the process proceeds to A&M 120 wherein sub-processes of Assessment 122 and Performance Goals 124 are undertaken. Within Assessment 122 the data obtained within the M&C 110 step are analysed, for example, through their entry into a human body (anatomical) model (HBM) to define a series of two-dimensional (2D) and/or three-dimensional (3D) perspectives of the user's anatomy. These 2D and 3D perspectives and modelling/analysis with the HBM may exploit one or more machine learning processes and/or algorithms and/or systems. Additionally, other parameters and/or aspects including, but not limited to, the following may be included:
Bone structure definition;
Soft tissue structure definition;
Soft tissue strains;
Relative positions of bones and/or soft tissues and/or surrounding organs;
Static body position in one or more position such as supine, sitting, and standing, for example;
Dynamic body position such as walking, bending, squatting, lifting, and jogging, for example;
Dynamic forces and structural measurements; and
Dynamic pressure from activities such as cough and Valsalva, for example.

Within Performance Goals 124 the QoL 116 data is established as specific static and dynamic performance goals for the USTD. These may include, but not be limited to, whether the USTD is to address urinary and/or fecal incontinence, number of episodes and volume, degree of comfort level required, will or can the user perform self-removal/cleaning/insertion etc., will this require periodic visits to a physician or clinic, and will any coatings require the user periodically dispose of the USTD and use a new USTD. Additionally, additional characteristics may be established with respect to providing an antimicrobial coating, providing controlled pharmaceutical product release(s) such as combinations of estrogen and progesterone for contraception, estrogen for treatment of genitourinary syndrome of menopause, spermicide, proteins, regenerative medicine(s) or other drugs for the user. These together with the data from Assessment 122 are employed in defining the custom USTD for the user in terms of physical geometry, e.g. dimensions of any ring structure, knob, support etc. Additionally, the mechanical properties of the custom USTD are defined in respect of the flexibility, dimensional stability, installation/removal means, physical characteristics of the USTD such as smooth/contoured surfaces and/or regions, etc. as well as other aspects such as any locking and/or release mechanisms.

The accumulated data from the Analysis & Modelling 120 as defined within Assessment 122 and Performance Goals 124 is coupled to an Artificial Intelligence (AI) Engine 160 which employs a plurality of algorithms which may exploit one or more approaches including, but not limited to, those based on symbol manipulation, cognitive simulation, logic-based programming, anti-logic programming, natural language processing, knowledge based, sub-symbolic, embodied intelligence, computational intelligence and soft computing, and statistical either individually or in combination such as within methodologies such as the intelligent agent, multiple interacting agents in a multi-agent system, and a hybrid intelligent system.

The AI Engine 160 may employ a hierarchal control system to bridge between sub-symbolic AI and symbolic AI. Tools exploited by the AI Engine 160 may include, but are not limited to, search and optimization, evolutionary computation, swarm intelligence algorithms, evolutionary algorithms, logic programming, fuzzy systems, subjective logic, default logics, non-monotonic logics, circumscription, probabilistic methods for uncertain reasoning. Bayesian networks, Hidden Markov models, utility theory, decision theory, Kalman filters, dynamic decision networks, classifiers and statistical learning methods, classifiers, neural networks, kernel methods, k-nearest neighbour algorithm, naïve Bayes classifier, decision tree, neural networks, artificial neural networks, acyclic or feedforward neural networks, recurrent neural networks, perceptrons, multi-layer perceptrons, radial basis networks, backpropagation networks, deep feedforward neural networks, convolutional neural networks, reinforcement learning, deep recurrent neural networks, recurrent neural networks, and gradient descent training.

The output of the AI Engine 160 is coupled to Custom Device Manufacturing and Fitting (CUDEMAF) 130 which proceeds with a sequence comprising Manufacture 132 and Fitting 134. Within Manufacture 132 the custom USTD is defined in respect of the materials providing its physical geometry with the desired mechanical properties as well as external characteristics. Accordingly, the custom USTD may be defined by one or more aspects including, but not limited to:
Scaffold structure by dimension(s), material(s) etc.
Shell structure by dimension(s), material(s) etc.
Casing structure by dimension(s), property or properties, material(s).
Passive—active integration such as is USTD passive or does it embed sensor(s), control and/or data logging circuitry, wireless interface(s) etc.
Lock-release structure.
Coatings.

Accordingly, a CAD model is established from which the Manufacture 132 process is undertaken. Within an embodiment of the invention an initial CAD model may be established by combining three-dimensional (3D) modelling with computational fluid dynamics (CFD), finite element analysis (FEA), and/or multi-organ free-body diagram models. The CAD model may be simplified to reduce the required computational power and complexity of the processing applied prior to the AI Engine 160 executes. The AI Engine 160 may process based upon this initial pre-processing solely or may apply the pre-processing to a more complete human body (anatomical) model and USTD model in order to define the USTD design, CAD, and materials requirements. Optionally, the pre-processing may be bypassed where appropriate levels of computing resources are available. Within an embodiment of the invention the AI Engine 160 generates the specifications for design of the USTD in dependence upon the computational modelling, FEA analysis, 3D modelling either individually or in combination.

Accordingly, a USTD as designed and manufactured may range from a passive USTD through to an active USTD, with lock-release structure, anti-microbial coating, and wireless interface for transmitting and logging data relating to the user. An active USTD may incorporate one or more sensors or stimulation mechanisms, such as electrostimulation, for example.

Within Fitting 134 the custom USTD is provided to the user and either fitted by themselves, e.g. for temporary use USTD that the user will insert/remove as desired, or by a clinician, e.g. semi-permanent or permanent use. At this point one or more assessments may be carried out such as outlined previously in respect of Structural 112 and/or Force, Strain and Distension 114 whereby, for example, mechanical, imaging, static and/or dynamic assessment etc. are performed to assess the USTD fit against the target design/user physiology etc. Optionally, the Structural 112 and/or Force, Strain and Distension 114 may be device based assessments and/or non-device based (e.g. clinical) assessments. This stage may also include device-acquired user monitoring, e.g. via internal sensors to the USTD, as well as user-reported monitoring, e.g. by personally noting performance of the USTD etc. Based upon these results a determination is made as to whether the USTD meets the initial requirements wherein if yes, the process proceeds to step 140. If not, then the process proceeds to loop back to either A&M 120 or CUDEMAF 130 according to the nature and/or complexity of the modifications/amendments required.

In step 140 the user employs the USTD on an ongoing basis wherein device-acquired monitoring, e.g. via internal sensors to the USTD, as well as user-reported monitoring, e.g. by personally noting performance of the USTD etc. are performed wherein periodically this data is employed in determining whether the objectives for the USTD were met in step 150. If yes, then the process loops back to step 140 otherwise it proceeds back to step 110. For example, a young user may require multiple USTDs within the space of a few years/decade during their childhood, puberty, adolescence, etc. with evolving dimensions and requirements whereas an elderly user may require a single adjustment or no adjustment according to their circumstances.

Within the description supra monitoring of the user has been described and discussed with respect to the fitting, assessment, and performance monitoring of a USTD or USTD according to an embodiment of the invention. Whilst this may exploit one or more sensors embedded within the body of the USTD or upon its surface as discussed below it would be evident that the assessment may employ and exploit data acquired from a range of other wearable devices and biometric sensors in order to enhance, for example, the assessment, fitting, and monitoring of USTDs and/or USTDs according to embodiments of the invention wherein the additional data obtained, e.g. biometric data, environmental data, activity data, body position data, etc., provides correlation data and/or additional data For example, a patient suffering UI may experience incontinence when bending over and/or walking but not during sitting and/or being prone. Further, the ongoing acquisition of data from a range of other wearable devices and biometric sensors may also be employed in association with or without sensors within the USTD to provide ongoing quality of life (QoL) data to assess the effectiveness of the USTD.

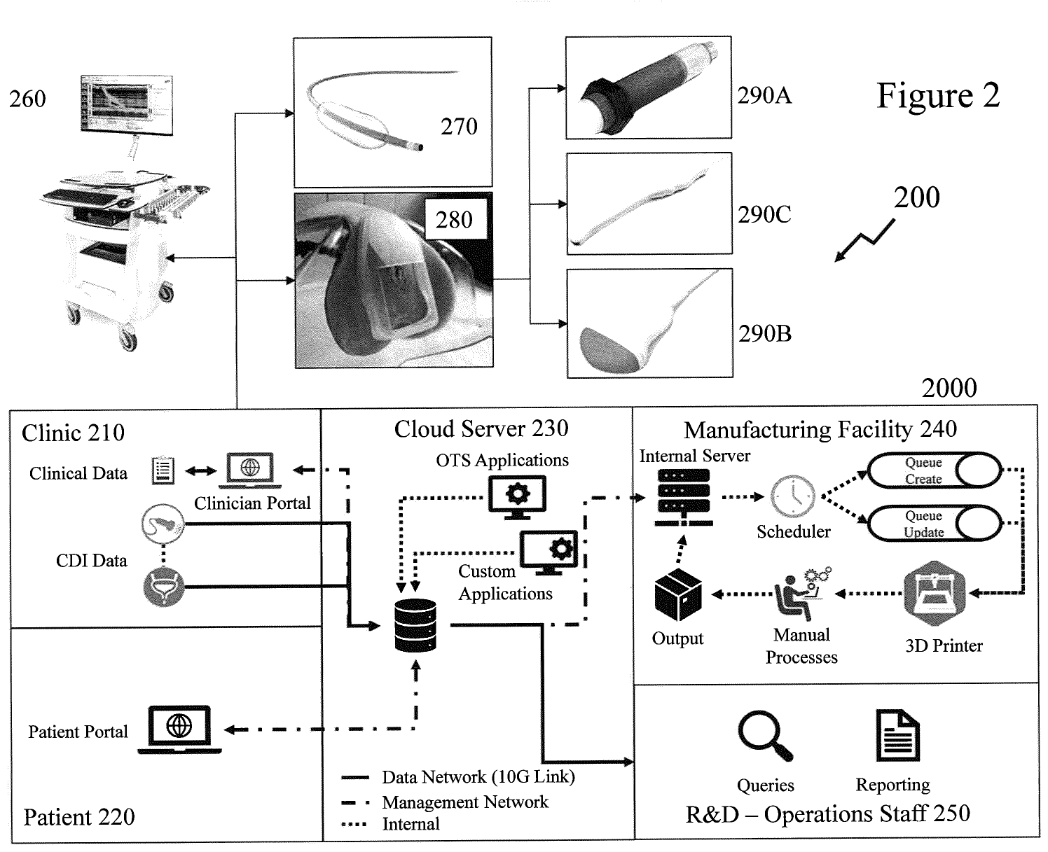

Now referring to FIG. 2 there is depicted an exemplary USTD Platform and System 200 supporting embodiments of the invention comprising an initial measurement stage involving Clinic 210 and Patient 220. The data from the measurement stage is analysed using Cloud Server 230 resulting in outputs to Manufacturing Facility 240 and R&D-Operations Staff 250. Considering Clinic 210 then this is depicted as comprising clinical data which is obtained via a Clinician Portal and stored upon the remote cloud server and clinical data integration (CDI) data which is automatically transferred from the measurement system(s) to the remote Cloud Server 230. This CDI data may include, but not be limited to colpodynamic imaging data. The clinical data may comprise clinician measurements, observations, assessments etc. of the patient such as described and depicted above with Manual measurements in Structural 112 of FIG. 1 for example together with data for Quality of Life 116 in FIG. 1. The CDI data, in contrast, is automatically acquired data relating to the user through one or more measurement systems such as Manometry System 260 together with Manometer Balloon 270 and an embodiment of the invention, a Manometry Cap 280, which is employed with a two-dimensional (2D) ultrasound Probe 290A or a three-dimensional ultrasound Probe 290B within embodiments of the invention. This CDI data is automatically pushed to the remote Cloud Server 230.

Within Patient 220 the user through a Patient Portal enters data relating, for example, to Quality of Life 116 in FIG. 1. Subsequent to the user being fitted with the USTD then the user through the Patient Portal and the clinician through the Clinician Portal may each enter additional comments, observations, notes etc. with respect to the monitoring of the quality of life/performance of the USTD such as within step 140 of FIG. 1.

The data once stored upon the remote Cloud Server 230 from the Clinic 210 and Patient 220 is employed by a combination of Off-the-Shelf (OTS) applications and custom applications to generate the data required for the Manufacturing Facility 240 as well as any communications required to the R&D-Operations Staff 250. The remote Cloud Server 230 based upon the processing by the OTS applications and custom applications may also send communications to the Clinic 210 and the Patient 220. Data provided to R&D-Operations Staff 250 may include data for reporting and/or queries. The data provided to the Manufacturing Facility 240 from the remote Cloud Server 230 would be stored upon an internal server of the Manufacturing Facility 240 and would comprise USTD tracking data to associate the USTD to the Patient 220, manufacturing data for the USTD specific to the Patient 220, etc. This is then employed to schedule the manufacturing and control the manufacturing process using additive and/or non-additive manufacturing processes. The Cloud Server 230 may include an AI Engine, such as AI Engine 160 in FIG. 1.

After QA processes etc. the Clinic 210 and Patient 220 are advised the USTD has been manufactured and the USTD shipped to the Clinic 210 or the Patient 220. Accordingly, measurements from one or more measurement techniques including, but not limited to, the Manometry System 260 together with Manometer Balloon 270 and a Manometry Cap 280, which is employed with an instrument such as two-dimensional (2D) ultrasound Probe 290A,a three-dimensional ultrasound Probe 290B, or endoscopic Probe 290C for example, are employed by the Cloud Server 230 to generate the USTD specific manufacturing data including dimensions, design, material(s), etc. employed by the Manufacturing Facility 240.

Accordingly, the USTD Platform and System 200 provides for collection of data from several sources including, but not limited to:
a clinician application or clinician portal (which may integrate with a clinic's Electronic Medical Records (EMR) software suite or cloud services, Electronic Health Records (EHR) software suite or cloud services, etc.:
a patient application or portal; and
a provider of USTDs (e.g. COSM Medical) clinical data integration (CDI) software application or cloud services.

Accordingly, the USTD Platform and System 200 stores data within one or more cloud services wherein this stored data is exploited to provide analysis and prediction software methods and applications including, but not limited to:
Anatomy identification
3D segmentation and key metric extraction;
2D video processing for dynamic analysis and/or analysis of pressure, force, direction and/or magnitude; and
USTD device geometry and dimensions using one or more prediction models.

Accordingly, several software applications and/or portals as depicted within USTD Platform and System 200 transfer data to/from the Cloud Server 230 (herein referred to as Cloud Infrastructure). Accordingly, the Cloud Infrastructure supports a USTD Platform and System 200 as well as other USTD platforms and systems according to embodiments of the invention. For example, a Clinician may access a Clinician Portal which provides data to and/or receives data from one or more Web Servers which may or may not include Cloud Server 230 for example. These Web Servers being in communication with one or more Web Applications which interface with, for example, a Clinical Remote Desktop Services (RDS) Server. Similarly, a Patient accesses a Patient Portal which similarly communicates with the Web Servers.

The acquisition of patient specific data, such as physical measurements, 2D or 3D scans etc., are processed by an Imaging or Clinical Data Integration (CDI) process wherein acquired imaging and/or clinical data is communicated to a Cloud Server instance before Data Validation is undertaken where the acquired data is processed to ensure it is valid before it is stored within one or more Data Lakes. The CDI may, for example, communicate directly with Data Validation via secure links, data tunnels, etc. as known in the art. Optionally, this data may also be pushed via one or more Web Servers. The data stored within the Clinical RDS and Data Lakes is archived/backed up within Cold Storage and Backup for example. The data from Clinical RDS and Data Lakes may also be accessed by Analysis Applications representing yet further Cloud Server instances.

One or more Analysis Applications may access the Clinical RDS and other RDS resources storing data including, but not limited to, analysis software, design databases, material databases, quality of life databases etc. The Analysis Applications or a specific application within the Analysis Applications may also communicate with an E-Commerce Portal allowing a Clinician to initiate an analysis, design, and manufacturing process through an e-commerce process. During this e-commerce process the Clinician may be provided with data prior to their review and approval of the manufacturing process. This data may include, but not be limited to, the USTD device geometry and dimensions, patient data, patient quality of life data, CDI data and predictions of USTD quality of life. Similarly, these other RDS resources may be archived/backed up within Cold Storage and Backup.

Within embodiments of the invention the E-Commerce Portal provides one or more functions (features) to a clinician/physician as well as to other third parties. For example, the E-Commerce Portal may provide for:
A clinical decision confirmation system (CDCS) providing:
   Recommended USTD designs;
   Three-dimensional visualization of patient's physiology and USTD device;
   Ability for clinician to edit and update key features and/or dimensions;
   Clinician approval of the design for manufacturing;
Transmittal of physician approved USTD device to manufacturing system and/or USTD device provider:
Payment processing including, for example, billing clinic, patient, and insurance;
Shipment tracking
Notifications;
USTD device fitting appointment scheduling; and
A telemedicine platform for post USTD device fitting care pathway.

As outlined above commercially available off-the shelf pessaries according to the prior art are limited on shape and specific size. As discussed above in respect of FIGS. 1 and 2, as well as outlined within World Intellectual Property Office patent application 2019/051,579 or U.S. Provisional Patent Application 63/133,913, both entitled “Methods and Systems for Vaginal Therapeutic Device Fitting”, the inventors have established methods, processes, systems and devices to provide patient-matched pessaries for each patient's (user's) unique anatomy and symptoms. The input data for personalization may include, but is not limited to, clinician provided anatomical measurements, such as Pelvic Organ Prolapse Quantification (POP-Q), obtained through physical examination, two-dimensional (2D) and/or three-dimensional (3D) imaging measurements, patient reported outcomes such as provided via questionnaires etc., or data from novel techniques such as the inventive colpodynamic imaging (CDI) method(s) established by the inventors.

POP-Q is a standardized tool used by clinicians as the current standard-of-care in assessing and staging pelvic organ prolapse. However, the inventors are establishing a CDI method that integrates 3D pelvic ultrasound imaging with vaginal pressure-volume measurements (such as obtained using a balloon and catheter connected to a urodynamic system for example). Transperineal ultrasound images are obtained using a 3D ultrasound probe wherein, for example, the vagina has been distended using a non-latex female condom inserted in the vagina and gradually filled with sterile water through a catheter as part of a urodynamic system until the vaginal capacity is reached. The outcome of the CDI procedure there may comprise ultrasound images, pressure and volume data from the vaginal cavity, through static (at rest) and dynamic maneuvers (e.g. contraction and Valsalva). The analysis of CDI data yields measurements related to the geometry of the vagina (referred to by the inventors as vaginal structural measurements (VSMs)).

These measurements then form part of a dataset used to determine the pessary shape and size based upon, for example, applying prediction algorithms to one or more parametric models. Each parametric model relates to a design or designs of a pessary which may be a prior art pessary shape or a novel inventive design according to an embodiment of the invention within a predetermined size range with additional customized parameters for the parametric model to optimize the fit, support, efficacy and comfort of the USTD over currently available off-the-shelf pessaries. The final form and dimensions of the USTD may be approved by a clinician before it is sent for manufacturing thereby bypassing the current risk management process where a clinician performs a trial-and-error fitting process for the pessary before a patient goes home with the device.

Within an exemplary embodiment of the invention the designed USTD may formed by the injection of a biocompatible liquid silicone rubber (LSR) into a 3D printed mold of the USTD or directly formed from the biocompatible LSR through an appropriate 3D printing technology. When fully cured, the USTD is coated with a biocompatible low coefficient of friction silicone coating before the USTD is cleaned, inspected, packaged, labelled, and shipped to the prescribing clinic where the physician performs the fitting process with the patient. Optionally, the USTD may be laser marked with identity data. Optionally, as outlined within World Intellectual Property Office patent application 2019/051,579 or U.S. Provisional Patent Application 63/133,913, both entitled “Methods and Systems for Vaginal Therapeutic Device Fitting” and the USTD may include other structural elements to provide the required performance which may be 3D printed from one or more other materials prior to application of an outer low friction coating.

It would be evident that the USTD may be designed based upon executing a range of parametric models, each relating to a different USTD design, in order to establish a design for the user where the selected design is one yielding, for example, a highest score in terms of one or more factors such as meeting QoL data, meeting performance objectives, cost, minimum design modifications, etc.

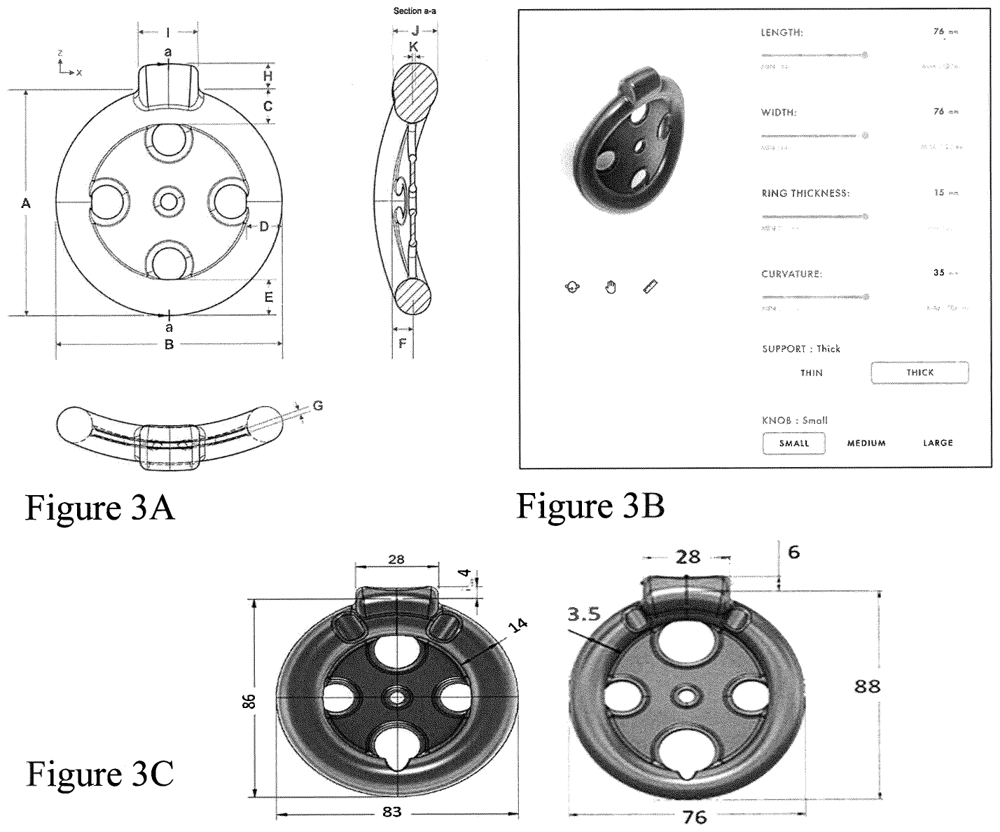

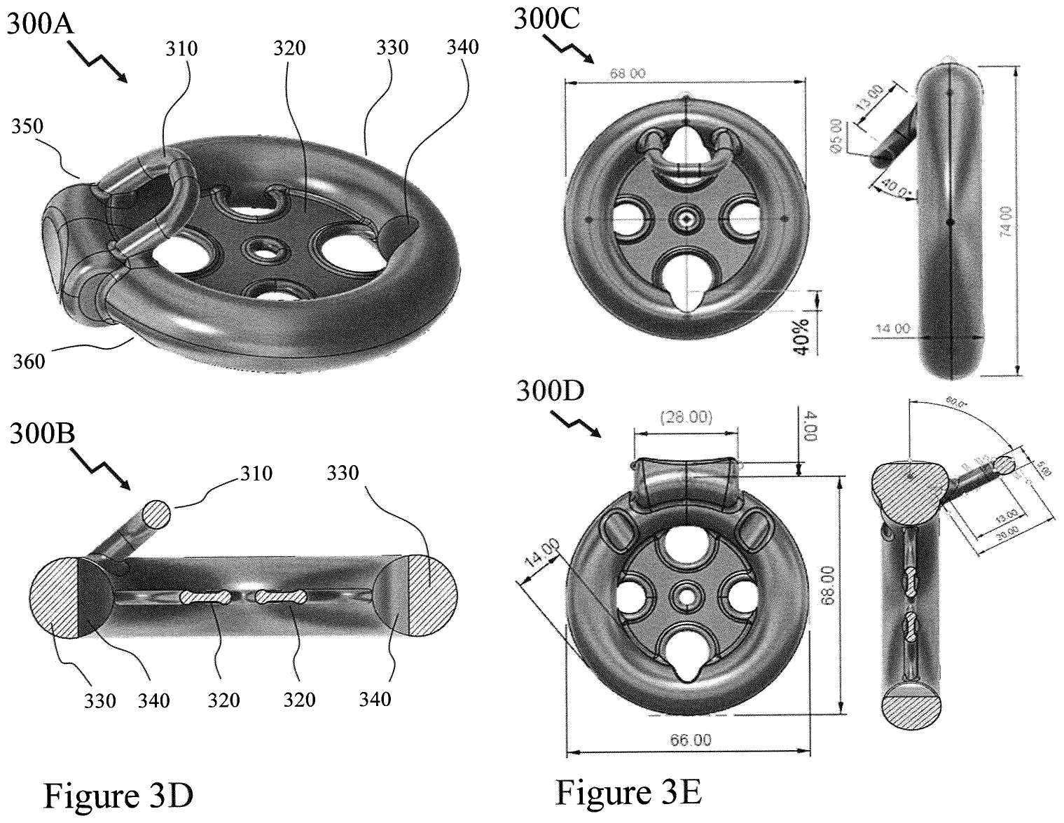

FIGS. 3A to 7B depict exemplary USTD designs which may provide parametric models for the analysis and design of the USTD for a specific user. However, it would be evident that other pessary design methodologies may be employed for parametric models without departing from the scope of the invention.

Referring to FIGS. 3A and 3B there are depicted physical geometry design parameters and exemplary design graphical user interface (GUI) for specifying a ring-type USTD according to an embodiment of the invention. FIG. 3A depicts the dimensions of the ring-type USTD according to embodiments of the invention which may be specified via a process established by the inventors, such as described for example with respect to World Intellectual Property Office patent application 2019/051,579 or U.S. Provisional Patent Application 63/133,913; either automatically and/or clinician input and/or modification. FIG. 3B depicts an exemplary design GUI for a clinician where rather than specifying all parameters they are presented with options, such as thick or thin support for example or small, medium or large knob for example, wherein the USTD software establishes the specific design parameters based upon the option selected. Accordingly, a clinician may simply review a design generated by software and/or be part of the design process.

Exemplary ranges for the identified dimensions depicted in FIG. 3A are presented below in Table 1 together with the exemplary factor(s) leading to the determination of the parametric variable for the patient (user). Referring to FIG. 3C there are depicted exemplary first and second ring-USTDs according to designs employed by the inventors within user trials.

TABLE 1
Exemplary Ring-Type USTD Dimensions

| | Minimum | Maximum | Parametric Variable Determined |
|---|---|---|---|
| Dimension | (mm) | (mm) | From |
| A | 44 | 127 | POP-Q + (standard method or |
| B | 44 | 127 | Measurement Tool) + CDI |
| C | 5 | 30 | POP force from CDI + mechanical |
| D | 5 | 30 | test data + SMART Fitting USTD |
| E | 5 | 30 | data |
| F | 0 | 70 | Mechanical test data (insertion/ |
| | | | removal versus support) + pessary |
| | | | orientation + SMART Fitting USTD |
| G | 1.5 | 30 | Mechanical test data (e.g. 3D |
| | | | force/displacement map) |
| H | 0 | 25 | POP-Q + (standard method or |
| | | | Measurement Tool) + CDI + 2D |
| | | | Image for urethra location + 2D |
| | | | video and SMART Fitting USTD |
| I | 0 | 45 | POP-Q + (standard method or |
| J | 0 | 70 | Measurement Tool) + CDI |
| K | −20 | 20 | |

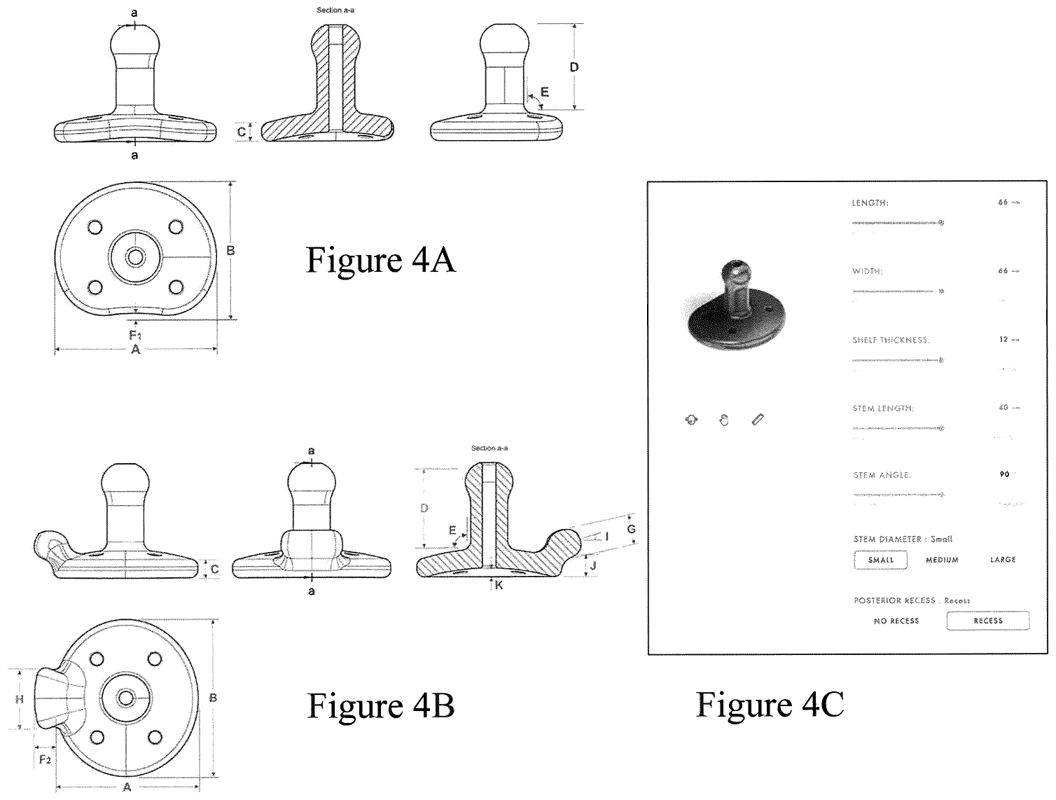

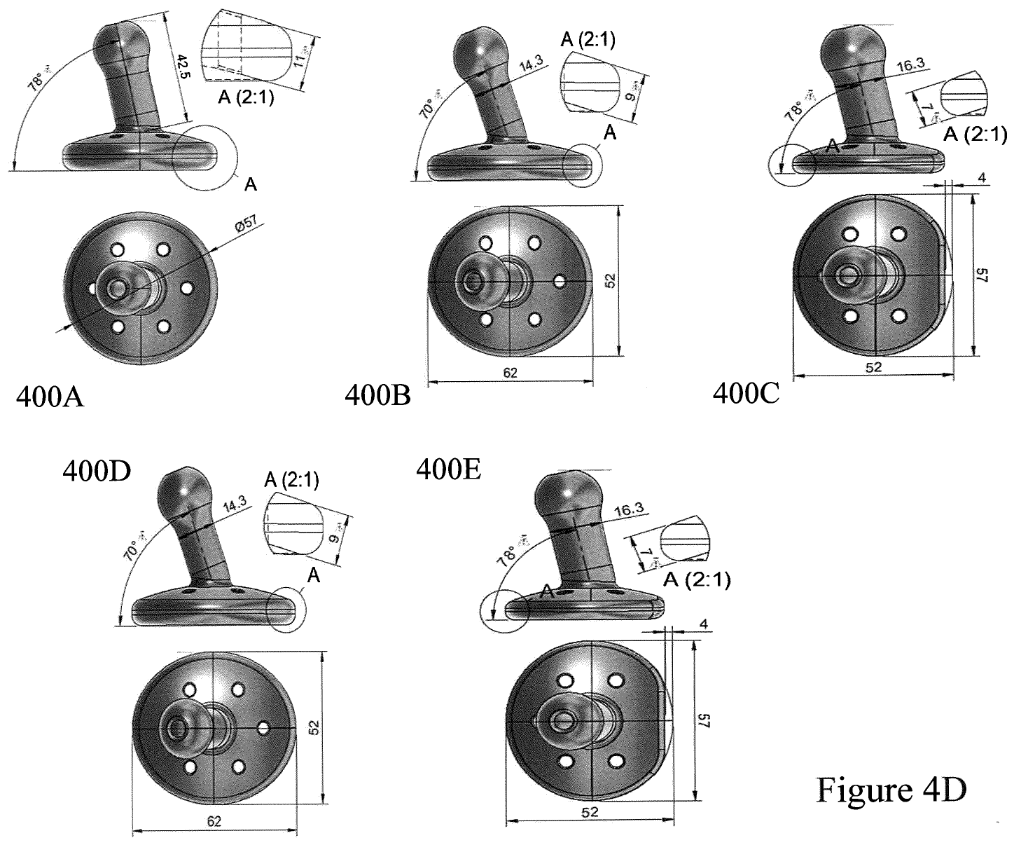

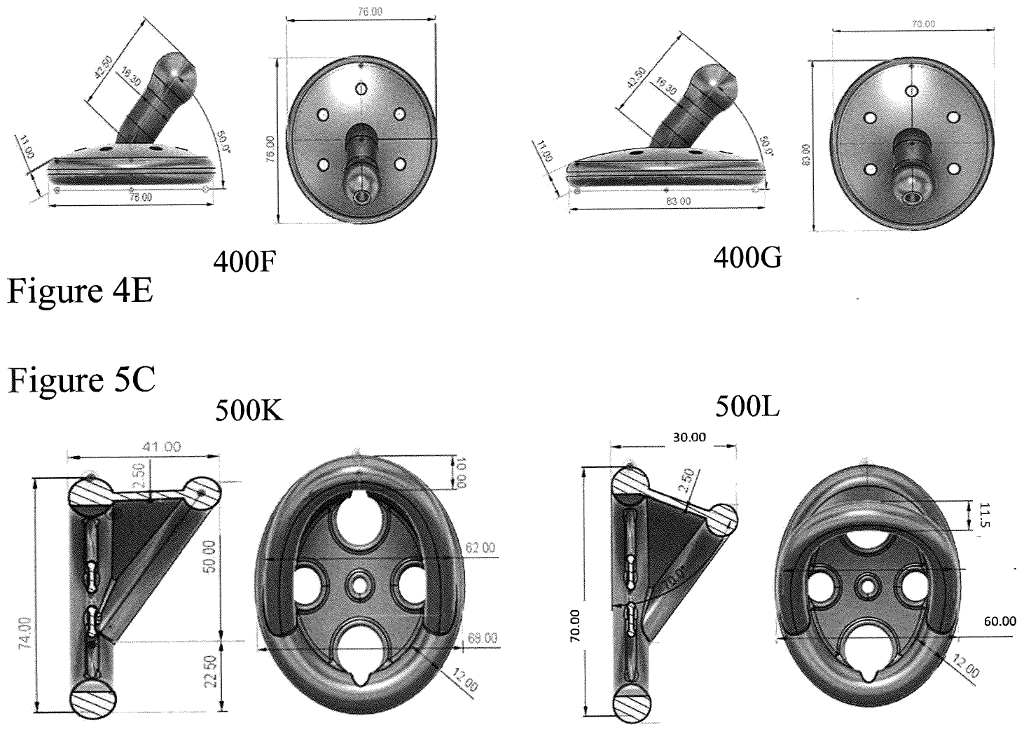

Now referring to FIG. 3D there are depicted first and second Ring Pessaries 300A and 300B according to embodiments of the invention wherein a “pull-tab” is provided to improve their usability by allowing the pull-tab to be employed in the insertion and/or removal of the first and second Ring Pessaries 300A and 300B respectively. First Ring Pessary 300A is depicted in perspective view and comprises a Pull Tab 310 which is connected to a Knob 350 which is disposed at upon the periphery of the Ring 330. A Support 320 is disposed on the inside of the Ring 330. The Ring 330 having a series of Notches on the inner periphery of the Ring 330 and lower surface of the Ring 330, where the upper surface of the Ring 330 is defined as that above which the Pull Tab 310 projects, which are depicted as first and second Notches 340 and 360 respectively. Second Ring Pessary 300B depicts a cross-sectional view of a ring pessary without a knob. Accordingly, the second Ring Pessary 300B comprises a Ring 330, Pull Tab 310 and Support 320 with Notches 340 on the inner periphery of the Ring 330. Optionally, the first and second Ring Pessaries 300A and 300B may be implemented without the Support 320. Referring to FIG. 3E there are depicted third and fourth Ring Pessaries 300C and 300D according to embodiments of the invention with a “pull-tab” provided to improve their usability by allowing the pull-tab to be employed in the insertion and/or removal of the third and fourth Ring Pessaries 300C and 300D respectively. Third and fourth Ring Pessaries 300C and 300D being according to designs employed by the inventors within user trials. Fourth Ring Pessary 300D has the “pull-tab” projecting to the exterior of the pessary rather than third Ring Pessary 300C where it projects to the interior of the pessary. Further, third Ring Pessary 300C has a “pull-tab” allowing the attachment of a pulling means, e.g. cord, hook, etc. whereas fourth Ring Pessary 300D has a solid “pull-tab” which is more suitable to a user's fingers for example. Now referring to FIGS. 4A to 4C there are depicted physical geometry design parameters and an exemplary design GUI for specifying Gellhorn-type USTDs according to embodiment of the invention. FIGS. 4A and 4B depict the dimensions of the Gellhorn-type USTDs according to embodiments of the invention which may be specified via a process established by the inventors, such as described for example with respect to World Intellectual Property Office patent application 2019/051,579 or U.S. Provisional Patent Application 63/133,913; either automatically and/or clinician input and/or modification. FIG. 4A depicting a Gellhorn-type USTD with knob whilst FIG. 4B depicts an asymmetric bodied Gellhorn-type USTD. FIG. 4C depicts an exemplary design GUI for a clinician where rather than specifying all parameters they are presented with options, such as thick or thin support for example or small, medium or large knob for example, wherein the USTD software establishes the specific design parameters based upon the option selected. Accordingly, a clinician may simply review a design generated by software and/or be part of the design process.

Exemplary ranges for the identified dimensions depicted in FIGS. 4A and 4B are presented below in Table 2 together with the exemplary factor(s) leading to the determination of the parametric variable for the patient (user). Referring to FIGS. 4D and 4E there are depicted exemplary first to seventh Gellhorn-type USTDs 400A to 400G respectively according to different designs employed by the inventors within user trials.

TABLE 2
Exemplary Gellhorn-Type USTD Dimensions

| | Minimum | Maximum | |
|---|---|---|---|
| | (mm) | (mm) | Parametric Variable Determined From |
| A | 38 | 95 | POP-Q + (standard method or Measurement |
| B | 38 | 95 | Tool) + CDI |
| C | 3 | 20 | POP force from CDI + mechanical test |
| | | | data + SMART Fitting USTD data |
| D | 25 | 55 | POP-Q + (standard method or Measurement |
| E | 45° | 135° | Tool) + CDI |
| F1 | 0 | 0.25 * A | POP-Q + (standard method or Measurement |
| F2 | 0 | 25 | Tool) + CDI + Mechanical test data |
| G | 0 | 25 | (insertion/removal versus support) + |
| H | 0 | 45 | pessary orientation + SMART Fitting USTD |
| I | −65° | 65° | |
| J | 0 | 45 | |
| K | 0 | 20 | 2D video + SMART Fitting USTD |

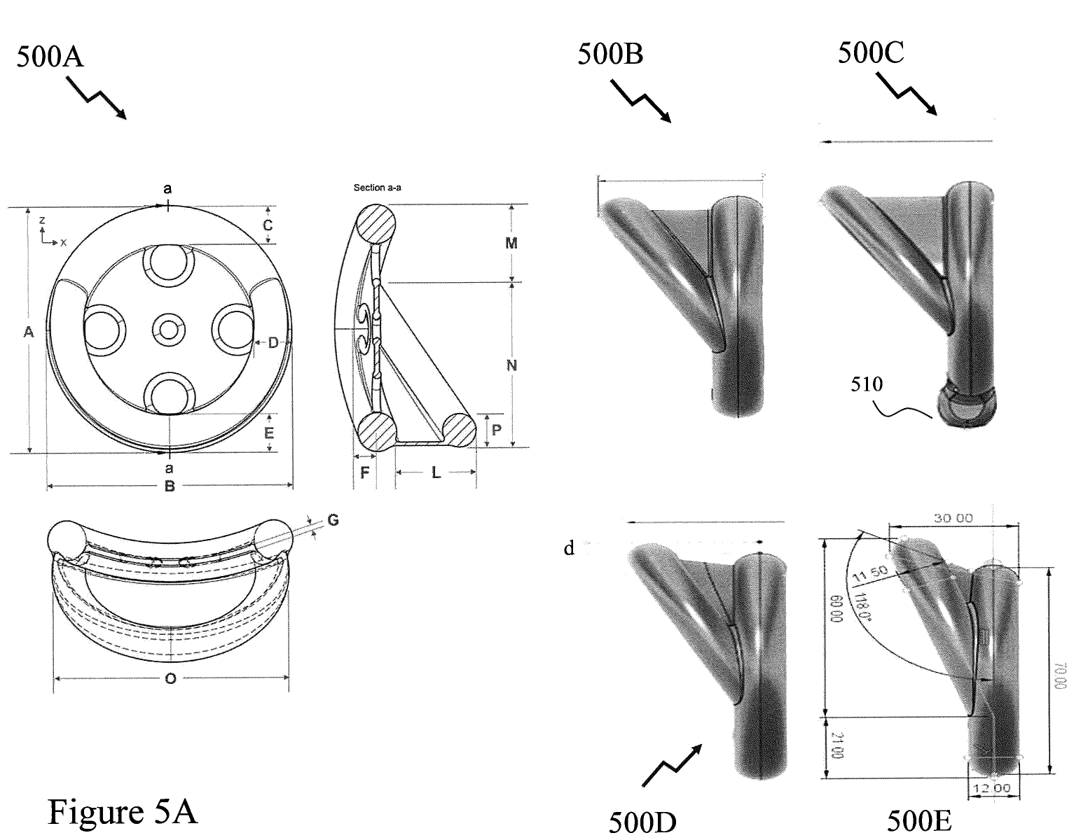

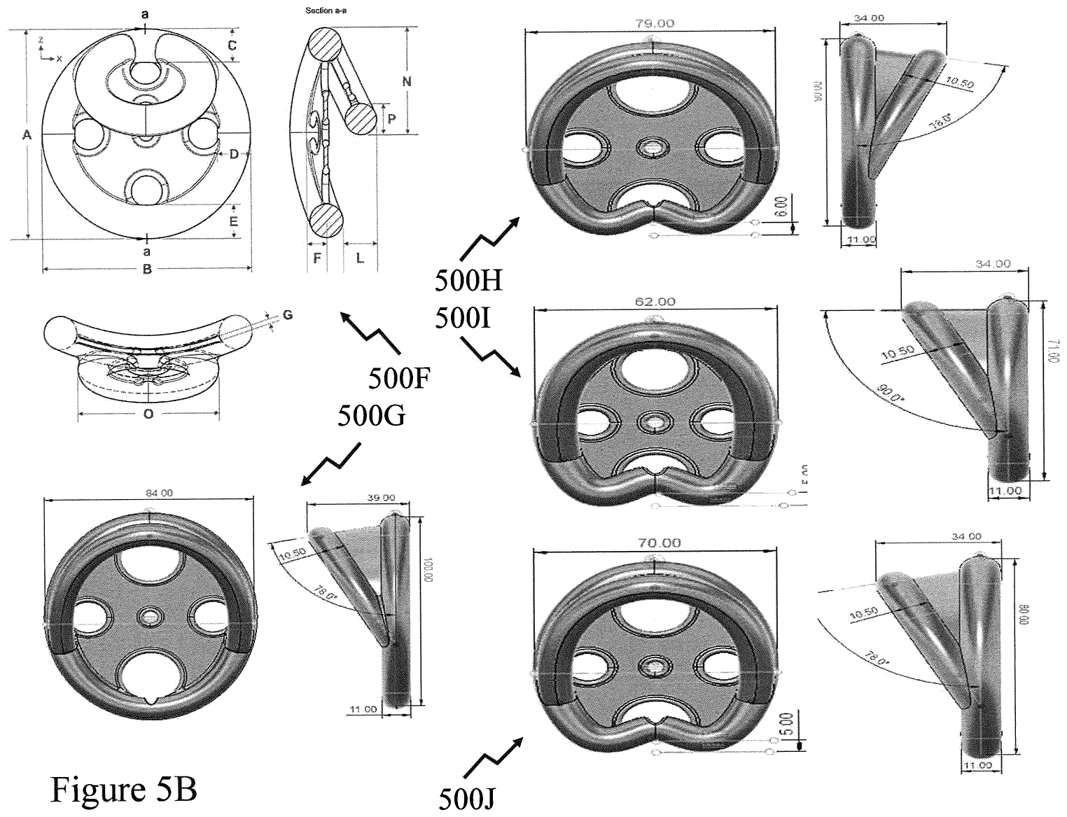

Now referring to FIG. 5A in first Image 500A and in fifth Image 500E there are depicted physical geometry design parameters for specifying Marland-type USTDs according to embodiment of the invention. First Image 500A in FIG. 5A and sixth Image 500F in FIG. 5B depict the dimensions of the Marland-type USTDs according to embodiments of the invention which may be specified via a process established by the inventors, such as described for example with respect to World Intellectual Property Office patent application 2019/051,579 or U.S. Provisional Patent Application 63/133,913; either automatically and/or clinician input and/or modification. Exemplary ranges for the identified dimensions depicted in first Image 500A in FIG. 5A and fifth Image 500E in FIG. 5A are presented below in Table 3 together with the exemplary factor(s) leading to the determination of the parametric variable for the patient (user).

TABLE 3
Exemplary Marland-Type USTD Dimensions

| | Minimum | Maximum | |
|---|---|---|---|
| | (mm) | (mm) | Parametric Variable Determined From |
| A | 44 | 127 | POP-Q + (standard method or Measurement |
| B | 44 | 127 | Tool) + CDI |
| C | 5 | 30 | POP force from CDI + mechanical test |
| D | 5 | 30 | data + SMART Fitting USTD |
| E | 5 | 30 | |
| F | 0 | 70 | POP-Q + (standard method or Measurement |
| | | | Tool) + Mechanical test data |
| | | | (insertion/removal versus support) + |
| | | | pessary orientation + SMART Fitting USTD |
| G | 1.5 | 30 | Mechanical test data (3D force/displacement |
| | | | map) |
| L | 10 | 85 | CDI + SMART Fitting USTD |
| M | 0 | 127 | CDI |
| N | 0 | 127 | |
| O | 0 | 127 | |
| P | 5 | 30 | POP force from CDI + mechanical test |
| | | | data + SMART Fitting USTD |

Second to fifth Images 500B to 500E in FIG. 5A and seventh to tenth Images 500G to 500J respectively in FIG. 5B depict exemplary Marland-type USTDs according to different designs employed by the inventors within user trials. Similarly, eleventh and twelfth Images 500K and 500L in FIG. 5C depict exemplary Marland-type USTDs according to different designs employed by the inventors within user trials. Referring to fourth and fifth Images 500D and 500E in FIG. 5A the upper ring portion of the Marland-type pessary projects outside the footprint of the lower ring portion by a distance, d, which may for example be a few millimeters (e.g. 5 mm). In contrast in twelfth Image 500L in FIG. 5C the upper ring portion of the Marland-type pessary is within the footprint of the lower ring portion. Within eleventh and twelfth Images 500K and 500L in FIG. 5C a wall portion exists between the lower ring portion and the upper ring portion whilst within other embodiments of the invention no wall portion may be implemented. An angle between the lower ring portion and the upper ring portion may be 90° or it may be less than 90°, e.g. 70° in twelfth Image 500L or greater than 90° as depicted in fourth and fifth Images 500D and 500E respectively in FIG. 5A.

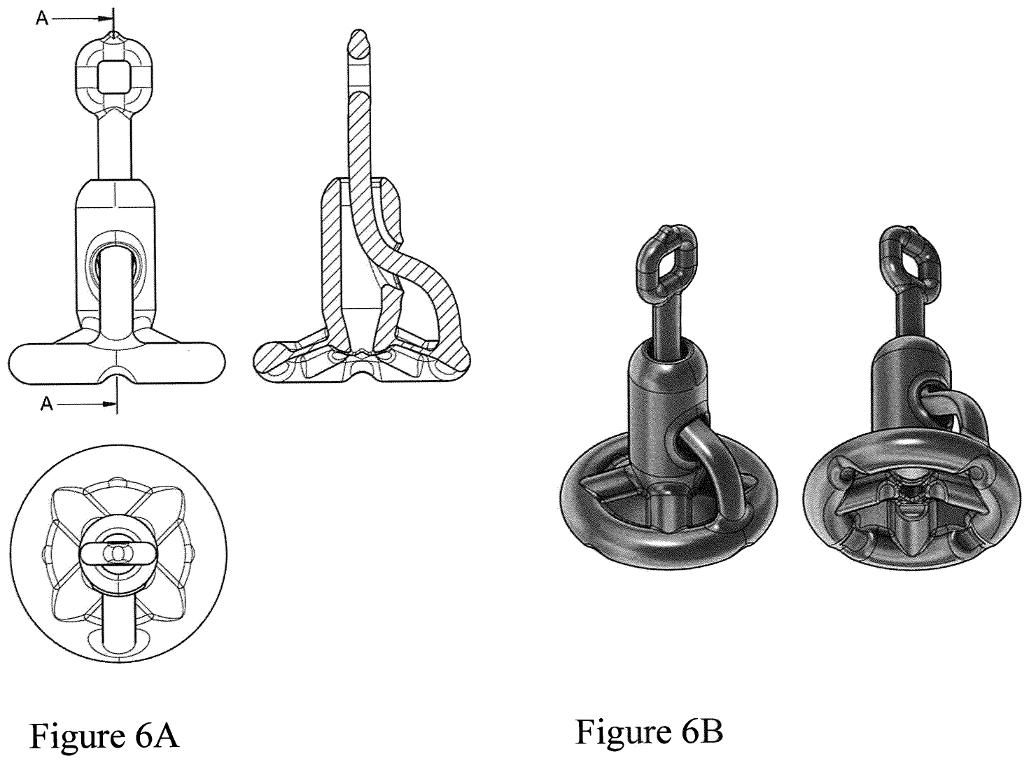

Now referring to FIGS. 6A and 6B there is depicted Gellhorn-type USTDs (GT-USTDs) according to embodiments of the invention with a first variant of a pull tab according to an embodiment of the invention. The inventors established that there were two drawbacks with the prior art Gellhorn design, variants of which are depicted in FIGS. 4A and 4B respectively. These were that the Gellhorn pessary is difficult to hold, manipulate and/or remove and that the pessary is difficult to fold when being manipulated into position for deployment. Accordingly, the inventors sought to establish alternate designs that addressed these issues. FIG. 6A depicts a front elevation, plan and cross-sectional side elevation along section line A-A for a GT-USTD according to an embodiment of the invention with a first variant of a pull tab. FIG. 6B depicts upper and lower perspective views of a 3D rendered model of a GT-USTD according to an embodiment of the invention with a first variant of the pull tab.

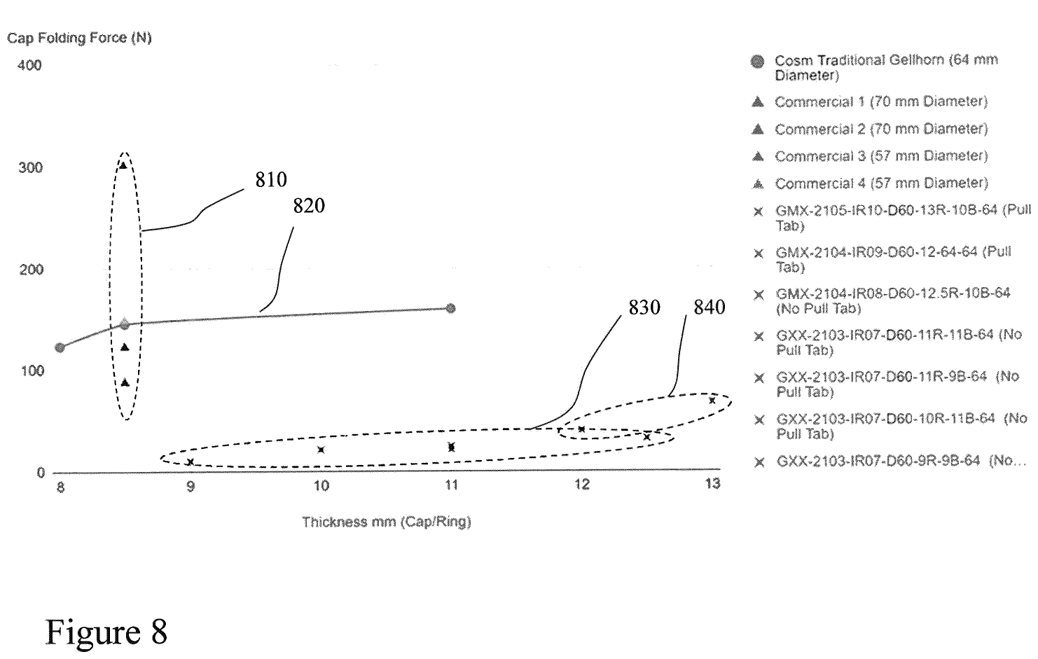

As depicted the GT-USTD comprises at its bottom an outer ring with a series of membranes and a plurality of ribs connected to the central shaft from the outer ring where each membrane is disposed between a pair of ribs. In contrast to the prior art Gellhorn pessary the GT-USTD has a pull-tab which extends down into the central shaft before exiting an opening within the central shaft and attaching to the outer ring. The end of the pull-tab distal to the ring having a loop element allowing attachment of a pull (e.g. a thread, string, floss, wire etc.). Accordingly, once deployed the pull-tab either directly or indirectly through a pull distorts the ring away from the user's skin such that any pressure differential either side of the outer ring/membrane retaining the GT-USTD in position is broken thereby making the GT-USTD easier to remove. Further, the hollow central shaft in conjunction with the membrane and ribs provides for a GT-USTD which is easier to bend than a prior art Gellhorn pessary as depicted from the experimental results in FIG. 8.

The lower surface of the outer ring is also depicted as having “notches,” including one aligned with the attachment point of the pull-tab to the outer ring such that contact of the outer ring with the user's body is broken faster and air can flow from one side of the GT-USTD to the other to remove any pressure differential providing retention force for the GT-USTD against the user's body.

Whilst the GT-USTD is depicted with a single connection from the distal end of the pull-tab to the outer ring it would be evident to one of skill in the art that two or more connections may be provided such that a pull force applied to the pull-tab results in force being applied to multiple points on the outer ring. Optionally, the central shaft may be omitted within other GT-USTDs according to embodiments of the invention.

Within embodiments of the invention the pull-tab and outer ring may be formed from different materials or they may be formed from a single material. Optionally, the pull-tab may end in an element which is disposed within the outer ring either locally to the point where the pull-tab meets the outer ring or around a portion of the outer ring. Optionally, the pull-tab itself may comprise a central element to provide the desired ability to pull the outer ring which is then coated in an outer coating.

Whilst the central shaft and outer ring are depicted as circular it would be evident that other geometries may be employed for each either discretely or in combination such as elliptical, regular polygonal, irregular polygonal, defined by a formula, etc. Optionally, the pull-tab may be one of a series of pull-tabs that each extend from a first end coupled to the outer ring through an opening in the central shaft and join the other pull-tabs either at their second distal ends or at an intermediate point before a single pull extends beyond. The second distal ends may join within the central shaft or above it for example.

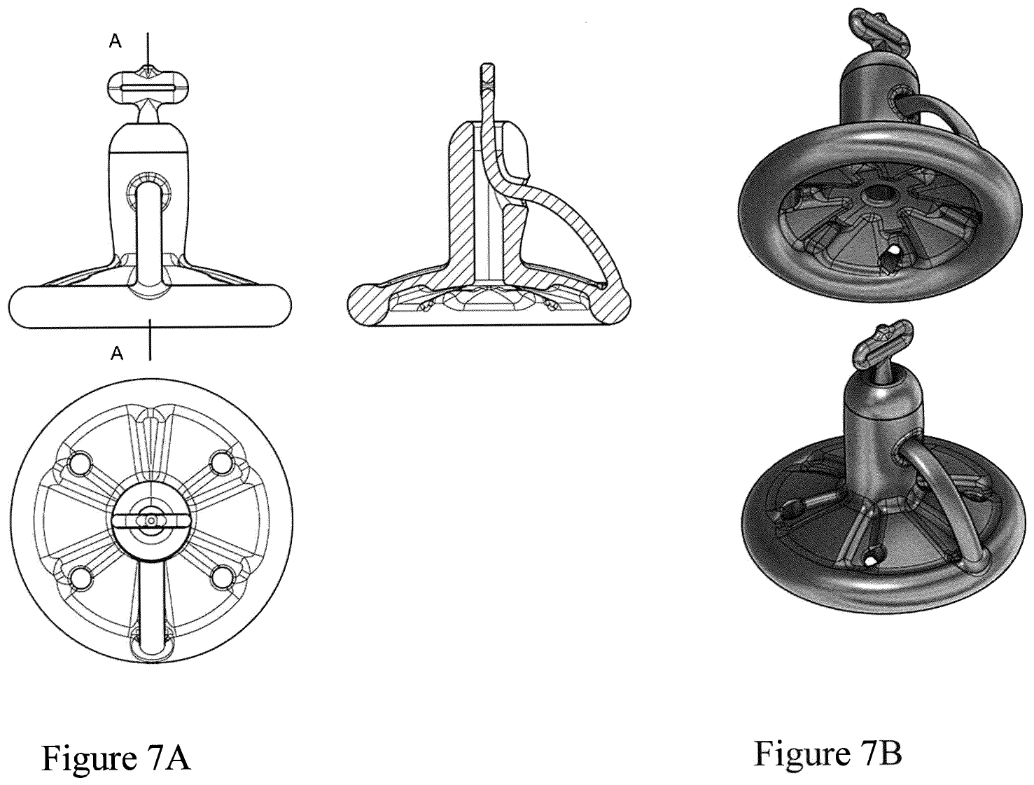

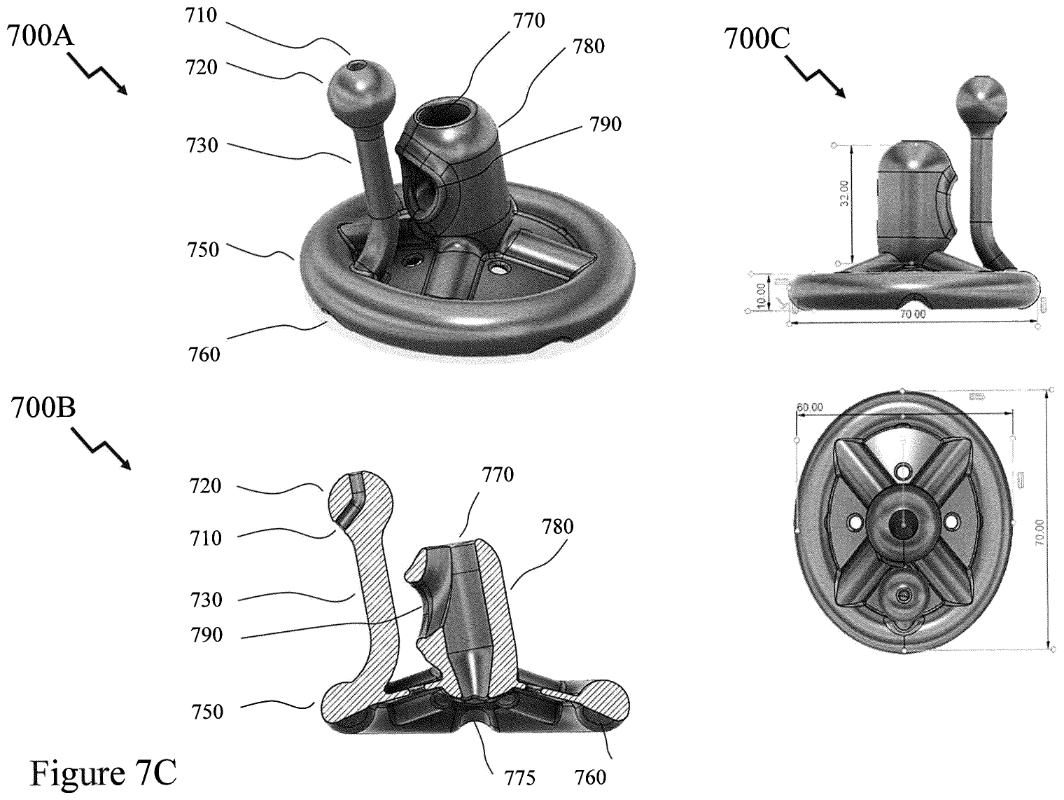

Referring to FIGS. 7A and 7B there are depicted a Gellhorn-type USTD according to an embodiment of the invention with a second variant of a pull tab according to an embodiment of the invention. FIG. 7A depicts a front elevation, plan and cross-sectional side elevation along section line A-A for a GT-USTD according to an embodiment of the invention with a first variant of a pull tab. FIG. 7B depicts upper and lower perspective views of a 3D rendered model of a GT-USTD according to an embodiment of the invention with a first variant of the pull tab.

Now referring to FIG. 7C there are depicted first and second Images 700A and 700B of a Gellhorn-Type Pessary (GT-Pessary) according to an embodiment of the invention wherein a “pull-tab” is provided to improve their usability by allowing the pull-tab to be employed in the insertion and/or removal of the GT-Pessary. Referring to first Image 700A there is depicted a perspective view of the GT-Pessary comprising a Ring 750 which has disposed at its centre a Tubular Horn 780 which is connected to the Ring 750 by a series of ribs between which are planar supports (these not being indicated by reference numerals for clarity). The Tubular Horn 780 projects to one side of the Ring 750 and has an Upper Opening 770 disposed at a distal end to that connected to the Ring 750 by the series of ribs. A Side Opening 790 is also disposed within the sidewall of the Tubular Horn 780. Also connected to the Ring 750 is Pull Tab 730 wherein another distal end of the Pull Tab 730 to that connected to the Ring 750 comprises a Grip 720 within which is a Guide 710. Disposed upon a lower surface of the Ring 750 are one or more Notches 760 which may be omitted within other embodiments of the invention.

Now referring to second Image 700B in FIG. 7C there is depicted a cross-section of the GT-Pessary depicted in first Image 700A. Accordingly, there is depicted a Lower Opening 775 disposed towards an end closest to a plane of the Ring 750 within the Tubular Horn 780 which is fluidically connected to the Upper Opening 770 disposed at a second distal end of the Tubular Horn 780 and the Side Opening 790. Also evident from the sectioning of the Ring 750 are the Notches 760 on the lower surface of the Ring 750. It is also evident from second Image 700B that the Guide 710 runs through the Grip 720 from a first position to a second position such that the Guide 710 allows for the insertion of a thread, wire, string or other connection allowing the Grip 720 to be pulled, and therein the Pull Tab 730 and Ring 750, from a location remote from the GT-Pessary. The thread, wire, string or other connection may be attached to the GT-Pessary via the Guide 710 prior to its insertion however it would be evident that the Grip 720 and Pull Tab 730 may be used without any attachment via the Guide 710.

As depicted the GT-USTD comprises at its bottom an outer ring with a membrane and ribs to a central shaft. In contrast to the prior art Gellhorn pessary the GT-USTD has a pull-tab which extends down into the central shaft before exiting an opening within the central shaft and attaching to the outer ring. The end of the pull-tab distal to the ring having a loop element allowing attachment of a pull (e.g. a thread, string, wire etc.). Accordingly, once deployed the pull-tab either directly or indirectly through a pull distorts the ring away from the user's skin such that any pressure differential either side of the outer ring/membrane retaining the GT-USTD in position is broken thereby making the GT-USTD easier to remove. Further, the hollow central shaft in conjunction with the membrane and ribs provides for a GT-USTD which is easier to bend than a prior art Gellhorn pessary as depicted from the experimental results in FIG. 8.

The lower surface of the outer ring is also depicted as having “notches,” including one aligned with the attachment point of the pull-tab to the outer ring such that contact of the outer ring with the user's body is broken faster and air can flow from one side of the GT-USTD to the other to remove any pressure differential providing retention force for the GT-USTD against the user's body. Also depicted in FIG. 7C is third Image 700C comprising side and plan elevations of a GT-USTD according to a design employed by the inventors within user trials.

Whilst the GT-USTD is depicted with a single connection from the distal end of the pull-tab to the outer ring it would be evident to one of skill in the art that two or more connections may be provided such that a pull force applied to the pull-tab results in force being applied to multiple points on the outer ring. Optionally, the central shaft may be omitted within other GT-USTDs according to embodiments of the invention.

Within embodiments of the invention the pull-tab and outer ring may be formed from different materials or they may be formed from a single material. Optionally, the pull-tab may end in an element which is disposed within the outer ring either locally to the point where the pull-tab meets the outer ring or around a portion of the outer ring. Optionally, the pull-tab itself may comprise a central element to provide the desired ability to pull the outer ring which is then coated in an outer coating.

Whilst the central shaft and outer ring are depicted as circular it would be evident that other geometries may be employed for each either discretely or in combination such as elliptical, regular polygonal, irregular polygonal, defined by a formula, etc. Optionally, the pull-tab may be one of a series of pull-tabs that each extend from a first end coupled to the outer ring through an opening in the central shaft and join the other pull-tabs either at their second distal ends or at an intermediate point before a single pull extends beyond. The second distal ends may join within the central shaft or above it for example.

Now referring to FIG. 8 there is depicted a plot of cap folding force of commercial Gellhorn pessaries against user specific GT-USTDs according to embodiments of the invention.

The results in first region 810 represent the variation in cap folding force (CAP-FF) for prior art commercial Gellhorn pessaries with outer diameters of 57 mm and 70 mm respectively (2.25″-2.75″) which have a thickness of 8.5 mm (0.35″) for the cap (central shaft)/outer ring. Line 820 depicts the CAP-FF for GT-USTDs according to designs of the inventors such as depicted in FIG. 4A with varying thickness of the cap/outer ring over the range 8-11 mm (0.31″-0.43″). Accordingly, the prior art Gellhorn pessaries had CAP-FF ranging from ˜90N to 300N whilst the inventor's implementation of the Gellhorn as depicted with respect to FIG. 4 varied from ˜120N to ˜160N.

However, the results in second and third regions 830 and 840 represent GT-USTDs according to embodiments of the invention as depicted in FIGS. 6A and 6B without or with the pull-tap respectively. Accordingly, it is evident that the CAP-FF required varies from ˜10N to ˜80N as the cap/shaft dimension increases from 9 mm to 13 mm (0.35″-0.51″) indicating a substantial reduction in the required force which must be applied by the clinician or other individual inserting the innovative GT-USTD into the patient. Whilst the use of the pull-tab increased the CAP-FF even those according to the innovative design with 13 mm (0.51″) cap/ring dimensions required less force than the prior art designs with 8.5 mm (0.35″) showing the requisite desired improvement in ease of folding the innovative GT-USTDs relative to prior art Gellhorn pessaries. These designs implemented by the inventors having a common outer diameter of 64 mm (2.52″).

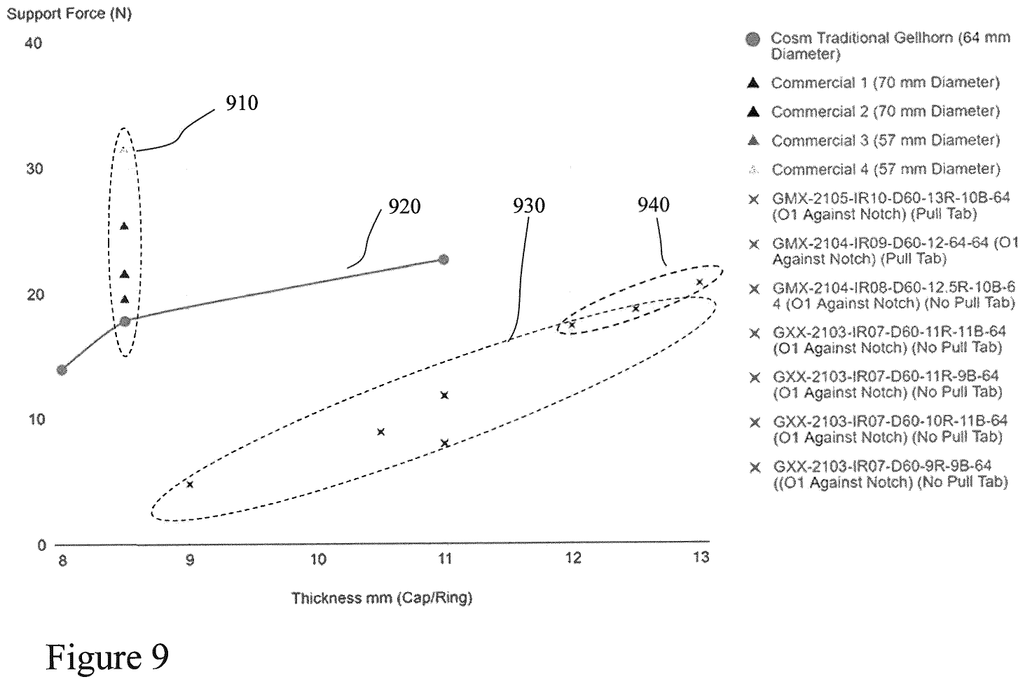

Referring to FIG. 9 there is depicted a plot of maximum bending force of commercial Gellhorn pessaries against user specific GT-USTDs according to embodiments of the invention. The results in first region 910 represent the variation in support force (SUP-F) for prior art commercial Gellhorn pessaries with outer diameters of 57 mm and 70 mm respectively (2.25″-2.75″) which have a thickness of 8.5 mm (0.35″) for the cap (central shaft)/outer ring. Line 920 depicts the SUP-F for GT-USTDs according to designs of the inventors such as depicted in FIG. 4A with varying thickness of the cap/outer ring over the range 8-11 mm (0.31″-0.43″). Accordingly, the prior art Gellhorn pessaries had SUP-F ranging from ˜19N to ˜31.5N whilst the inventor's implementation of the Gellhorn as depicted with respect to FIG. 4 varied from ˜13N to ˜22N.

However, the results in second and third regions 930 and 940 represent GT-USTDs according to embodiments of the invention as depicted in FIGS. 6A and 6B without or with the pull-tap respectively. Accordingly, it is evident that the SUP-F required varies from ˜5N to ˜20N as the cap/shaft dimension increases from 9 mm to 13 mm (0.35″-0.51″) indicating a substantial reduction in the required force which must be applied by the clinician or other individual inserting the innovative GT-USTD into the patient. However, the use of the pull-tab does not have any significant impact on the SUP-F. However, it is evident that the inventors GT-USTD design provides for adjustment of the SUP-F even for fixed outer diameter. These designs implemented by the inventors having a common outer diameter of 64 mm (2.52″). Further, designs with different outer diameters are expected to allow the overall trend to be shifted vertically such that different combinations of diameter and outer ring thickness can provide the same SUP-F whilst the overall range of SUP-F is increased relative to the prior art Gellhorn designs.

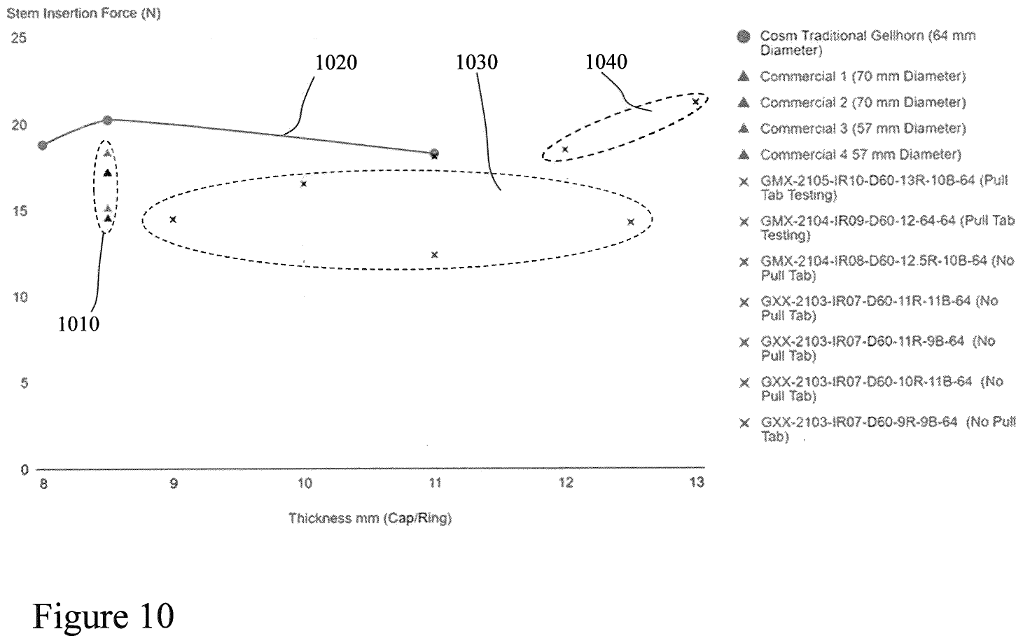

Now referring to FIG. 10 there is depicted a plot of stem insertion versus pull tab testing of commercial Gellhorn pessaries against user specific USTD Gellhorn devices according to embodiments of the invention. The results in first region 1010 represent the variation in stem insertion force (STEM-IF) for prior art commercial Gellhorn pessaries with outer diameters of 57 mm and 70 mm respectively (2.25″-2.75″) which have a thickness of 8.5 mm (0.35″) for the cap (central shaft)/outer ring. Line 1020 depicts the STEM-IF for GT-USTDs according to designs of the inventors such as depicted in FIG. 4A with varying thickness of the cap/outer ring over the range 8-11 mm (0.31″-0.43″). Accordingly, the prior art Gellhorn pessaries had STEM-IF ranging from ˜15N to ˜18N whilst the inventor's implementation of the Gellhorn as depicted with respect to FIG. 4 varied from ˜18N to ˜20N. However, the results in second and third regions 1030 and 1040 represent GT-USTDs according to embodiments of the invention as depicted in FIGS. 6A and 6B without or with the pull-tap respectively. Accordingly, it is evident that the STEM-IF required varies from ˜13N to ˜16N as the cap/shaft dimension increases from 9 mm to 13 mm (0.35″-0.51″) indicating a reduction in the required force. However, the use of the pull-tab resulted in increased STEM-IF of ˜18N-˜ 21N for outer ring diameters 12-13 mm (0.47″-0.51″) which is comparable to the results of line 1020.

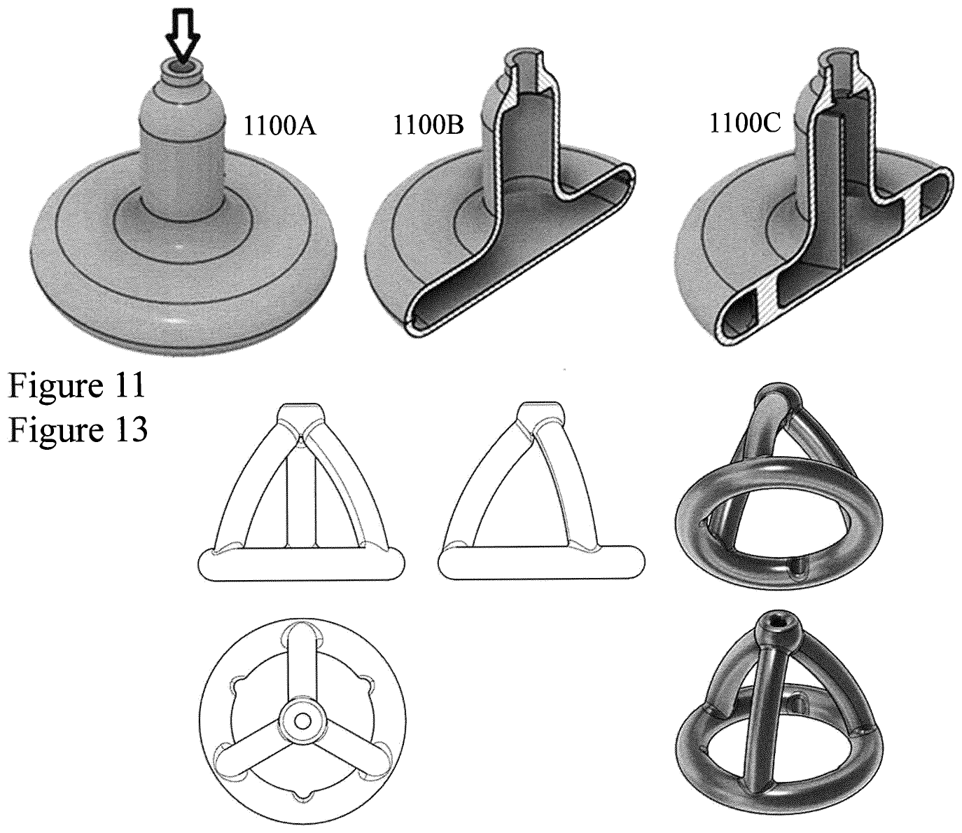

Referring to FIG. 11 there is depicted an inflatable GT-USTD (INF-GT-USTD) according to an embodiment of the invention. First and second images 1100A and 1100B depicted a perspective view and perspective cross-section view of a first design variant of the INF-GT-USTD whilst third image 1100C depicts a perspective cross-section view of a second design variant of the INF-GT-USTD. As depicted a fluid. e.g. air, nitrogen, water, etc., can be employed to fill and expand the INF-GT-USTD whilst a non-return valve (NRV, not depicted for clarity) either forming part of the stem of the INF-GT-USTD or piping connecting to the INF-GT-USTD prevents the fluid from escaping the inflated INF-GT-USTD until, for example, the NRV is physically manipulated to break the scal.

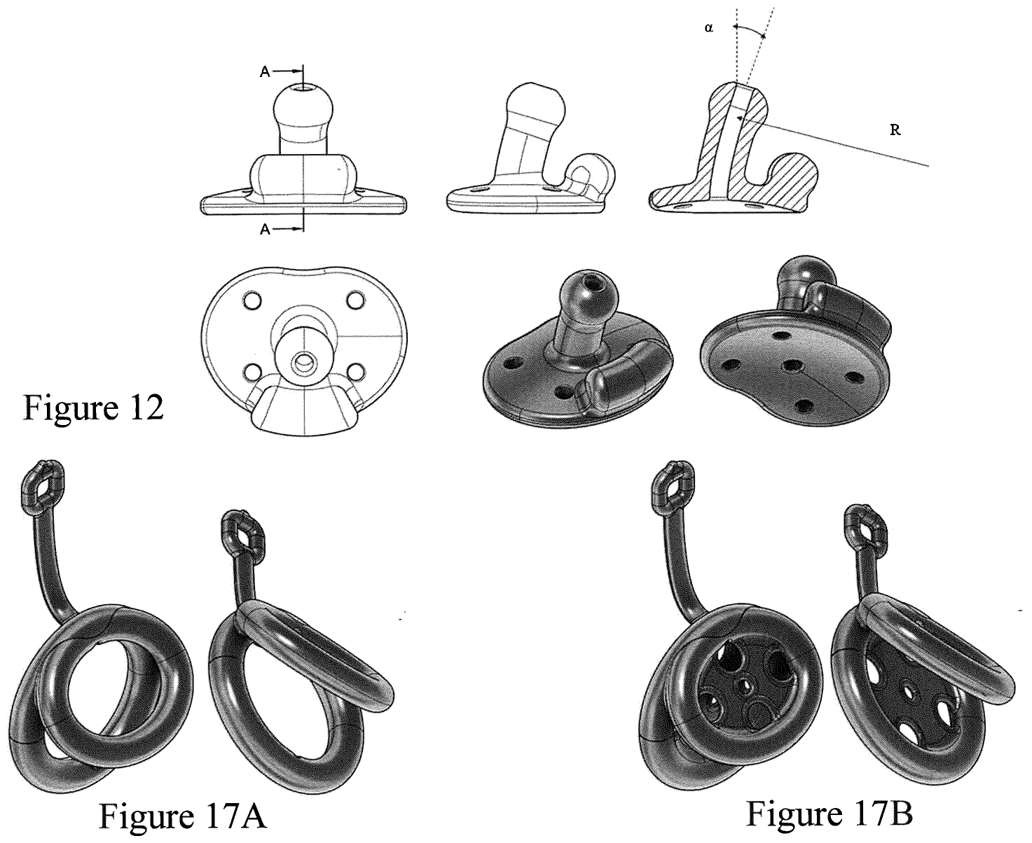

Optionally, the internal geometry of the INF-GT-USTD may be varied such as depicted in third image 1100C to include a ring fluidically isolated from the central inflatable portion of the INF-GT-USTD as well as mechanical elements disposed within the central inflatable portion of the INF-GT-USTD. This hollow outer ring is disposed around the periphery of the hollow body, fluidically isolated from the hollow body, and filled with another fluid to a predetermined at least one of a volume and a pressure. The another fluid can the same or different to the fluid employed to inflate the central inflatable portion of the INF-GT-USTD. Now referring to FIG. 12 there is depicted a GT-USTD with knob for urinary incontinence (UI) according to an embodiment of the invention. In contrast to the Gellhorn with knob designs depicted in FIGS. 4A and 4B the shaft of the GT-USTD where the shaft is perpendicular to the base of the pessary the shaft of the GT-USTD according to embodiments of the invention forms an arc of radius R and reaching a maximum angular deviation from the normal of a degrees. Within the embodiment of the invention depicted in FIG. 12 the shaft radially bends along an axis aligned with the knob and towards the knob. However, it would be evident that within other embodiments of the invention the shaft may radially bend along an axis aligned with the knob but away from the knob. Alternatively, the shaft may radially bend along an axis not aligned with the knob. Optionally, the shaft may radially bend with respect to two axes of the GT-USTD. Optionally, the design of the shaft may not be a section of circular arc but it may be a piece-wise linear fit to an arc, be defined by a polynomial or comprises two or more linear sections.

Referring to FIG. 13 there is depicted a collapsible pyramid type USTD (PT-USTD) according to an embodiment of the invention. Accordingly, the PT-USTD comprises an outer ring at its lower end with a series of arms connecting together at one end to ends disposed upon the outer ring. The number of “arms” may be two, three (as depicted), four or more. The “arms” as depicted are curved but within other embodiments of the invention these may be defined by a piece-wise linear fit to an arc, be defined by a polynomial, be linear or comprise two or more linear sections. Optionally, a pull-tab such as described and depicted with respect to FIGS. 6A-7B and 14A-17D may be employed.

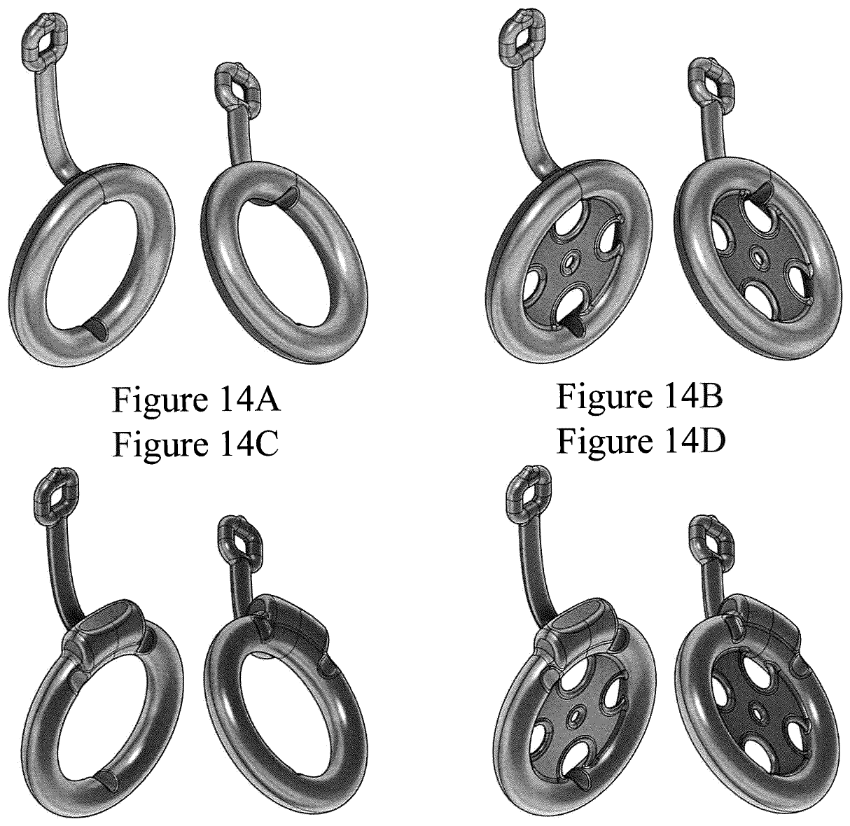

Now referring to FIGS. 14A-14D respectively there are depicted ring-type USTDs (RT-USTDs) according to embodiments of the invention with or without support and with or without knob configuration with a first pull tab variant. As depicted these comprise:
FIG. 14A is a RT-USTD with no support;
FIG. 14B is a RT-USTD with support in the central region of the outer ring;
FIG. 14C is a RT-USTD with a knob and no support; and
FIG. 14D is a RT-USTD with knob and support.

Referring to FIGS. 15A and 15B depict RT-USTDs according to embodiments of the invention with second and third pull tab variants. FIG. 15A depicts a RT-USTD with dual pulls attached to a pull-tab where the RT-USTD has a central support portion. Optionally, the pair of pulls may be attached to the outer ring after passing through holes within the central support. Optionally, the pair of pulls may be attached to the outer portion of the outer ring rather than the inner portion or one to the inner portion and one to the outer portion.

FIG. 15B depicts a RT-USTD with three pulls attached to a pull-tab where the RT-USTD has a central support portion. Optionally, four or more pulls may be attached. Optionally, the pulls may be attached to the outer ring after passing through holes within the central support. Optionally, the pulls may all be attached to the outer portion of the outer ring, all attached to the inner portion or some to the inner portion and others to the outer portion.

Now referring to FIGS. 16A and 16B there are depicted Marland-type USTDs (MT-USTD) according to embodiments of the invention with second and third pull tab variants. FIG. 16A depicts a MT-USTD without support whilst FIG. 16B depicts a MT-USTD with support. Optionally, two or more pulls may be coupled to a single pull-tab such as depicted with respect to the RT-USTDs in FIGS. 15A and 15B respectively with attachments to the inner portion and/or outer portion for each pull. Optionally, the pull may be attached to the other portion (partial ring) of the MT-USTD rather than the full ring or both where two or more pulls are employed.

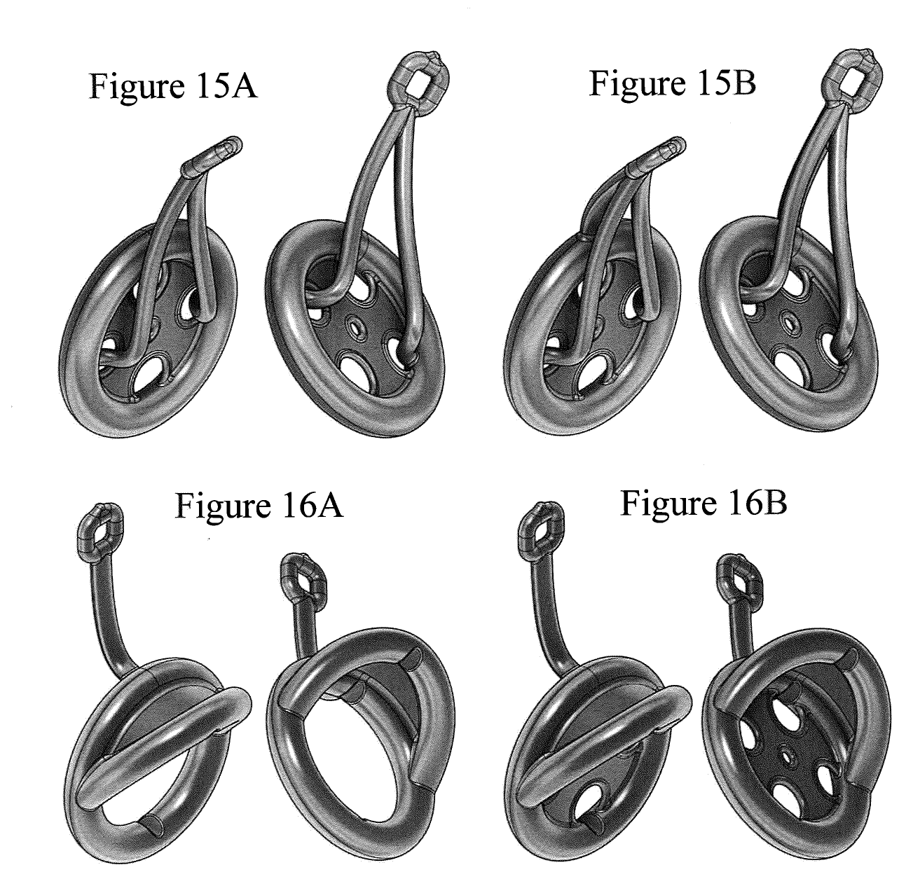

Referring to FIGS. 17A and 17B there are depicted V-shaped double ring-type USTDs (VDR-USTDs) according to embodiments of the invention with a pull tab. FIG. 17A depicts a VDR-USTD without support whilst FIG. 17B depicts a VDR-USTD with support. Optionally, two or more pulls may be coupled to a single pull-tab such as depicted with respect to the RT-USTDs in FIGS. 15A and 15B respectively with attachments to the inner portion and/or outer portion for each pull. Optionally, the pull may be attached to the other portion (i.e. smaller ring as depicted) of the VDR-USTD rather than the full ring or both where two or more pulls are employed.

It would be evident to one of skill in the art that the length of the pull tab(s) for the USTDs depicted in FIGS. 14A-17B respectively may be varied within other embodiments of the invention. Further, it would be evident to one of skill in the art that the position of the pull tab attachments(s) for the USTDs depicted in FIGS. 14A-17B respectively may be varied within other embodiments of the invention. For example, the length of the pull tab may be reduced such that the user does not feel the pull tab. The location and length of the pull tab may be established in dependence upon one or more factors including, but not limited to, the physiology of the user, the condition(s) affecting the user, and the performance objections of the USTD.

Pelvic floor disorders affect muscles and ligaments in the female pelvic floor which can have drastic negative impacts the sufferer's quality of life. Pelvic floor disorders may include urinary and/or fecal incontinence alongside pelvic organ prolapse (POP). POP treatment options include surgery, pelvic floor muscle training, and use of pessaries. Pessaries are removable vaginal prosthetics that come in 100+ shapes and sizes fit by trial and error. The inventors from the literature have established that using prior art pessaries about 1 in 3 sufferers fail pessary fittings, half of the pessary users will stop using them within 2 years, and over half will get complications with long term use. These statistics helped drive the inventors towards custom USTD methodologies that they have outlined above and within World Intellectual Property Office patent application 2019/051.579 or U.S. Provisional Patent Application 63/133,913.

Accordingly, whilst these documents disclose manual and automated means of obtaining measurements the inventors have established that prior art measurement devices suffer drawbacks in a similar manner as the standard prior art pessaries suffer drawbacks. Accordingly, the inventors have established a novel mechanical measurement tool to obtain more accurate dimensions for the pelvic floor exam results (POP-Q and POP-Q+). The objectives of the new tool being to remove subjectivity and increase consistency amongst clinicians. The inventors believe that using a novel measurement tool according to embodiments of the invention relative to prior art designs of such tools will increase the accuracy of pessary shape and size outputs from the POP-Q prediction algorithm(s) and therefore increase the success rate of custom USTD usage in POP patients.

Accordingly, the inventors have designed the new measurement tool (POPQ-Tool) to provide features for diameter measurement, length measurement, tactile feel of tissue distension and contact. With respect to diameter measurements the POPQ-Tool supports the measurement of the genital hiatus (GH, the distance between the urethral meatus and the posterior hymen spanning the anatomical area around the introitus), introitus diameter and the lateral spread. The lateral spread is measured with the POPQ-Tool past the first third of the total vaginal length (TVL) close to the cervix. The POPQ-Tool also allows for identification of and measurement of the widest point of the vagina.

With respect to length measurements the POPQ-Tool supports measurements of TVL, Diagonal Vaginal Length, Bp (uppermost point of the posterior vaginal wall), C (lowest edge of the cervix or the vaginal cuff), D (topmost point of the posterior vaginal wall), Aa (midline of anterior vaginal wall) and Ba (most superior location of the front vaginal wall).

During these measurements the POPQ-Tool has been designed to also facilitate the clinician having tactile feel of tissue distension, i.e. the POPQ-Tool provides a mechanism for feel and/or feedback to prevent over-distension of vaginal tissue. For example, where the POPQ-Tool employs a reverse-scissor type of mechanism (or similar if the POPQ-Tool is not shaped like scissors) wherein as the user closes the handle end of the tool, they are spreading the end effectors of the POPQ-Tool such that the user knows how much force they are applying to the vaginal tissue. Whilst embodiments of the invention may be spring-loaded the inventors have established primary designs which are not due to the wide range of force that can lead to over-distension of the vaginal tissue.

‘Within other embodiments of the invention one or more fingers of the medical personnel employing the POPQ-Tool may be inserted to contact the arms of the POPQ-Tool to provide an alternative method of tactile feedback during use of the POPQ-Tool.

The POPQ-Tool has also been designed in order to avoid or limit contact with anterior wall and anterior of the vagina at the introitus whilst performing lateral measurements. The POPQ-Tool is not intended to provide anterior-posterior measurements. It would also be beneficial for the POPQ-Tool to offer design options which are autoclavable, disposable and/or support disposable covers. Where disposable POPQ-Tools are established, it would be beneficial for these to employ materials such as Bamboo Biodegradable Material which can be molded/machined or other materials defined as biodegradable by organizations such as the United States Environmental Protection Agency (EPA) or combustible with no hazardous fumes. Where disposable it would be beneficial for the manufacturing cost points to be compatible with this. Similarly, if a disposable cover is employed that its cost be low and/or the disposable cover biodegradable or combustible with no hazardous fumes.

With respect to performance objectives for the POPQ-Tool the inventors established the inter-observer and intra-observer performance as defined in Table 4.

TABLE 4
Target POPQ-Tool Performance Objectives

| Measurement | Inter-observer | Intra-observer |
|---|---|---|
| Aa | 0.817, p < 0.0001 | 0.780, p = 0.0003 |
| | 0.89, 95% CI 0.79-0.99 | |
| Ba | 0.895, p < 0.0001 | 0.934, p < 0.0001 |
| C | 0.522, p = 0.0003 | 0.765, p = 0.0003 |
| | 0.72, 95% CI 0.48-0.96 | |
| D | 0.767, p = 0.0004 | 0.759, p = 0.02 |
| | 0.82, 95% CI 0.61-1.04 | |
| Ap | 0.747, p < 0.0001 | 0.826, p = 0.0001 |
| | 0.86, 95% CI 0.71-1.02 | |
| Bp | 0.746, p < 0.0001 | 0.859, p = 0.0001 |
| GH | 0.913, p < 0.0001 | 0.812, p = 0.0001 |
| PB | 0.514, p = 0.0004 | 0.659, p = 0.0002 |
| TVL | 0.488, p = 0.0008 | 0.431, p = 0.04 |

Ap: located midline of posterior vaginal wall 3 cm proximal to hymen
PB: perineal body recorded from the posterior aspect of hymen to the mid-anal opening

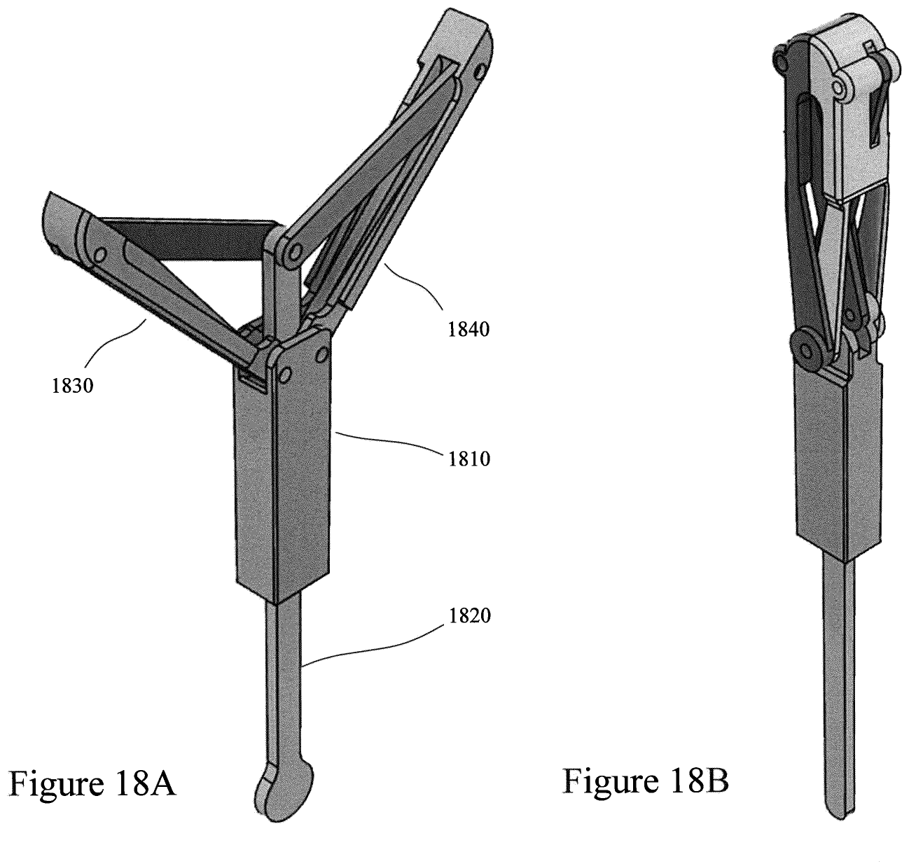

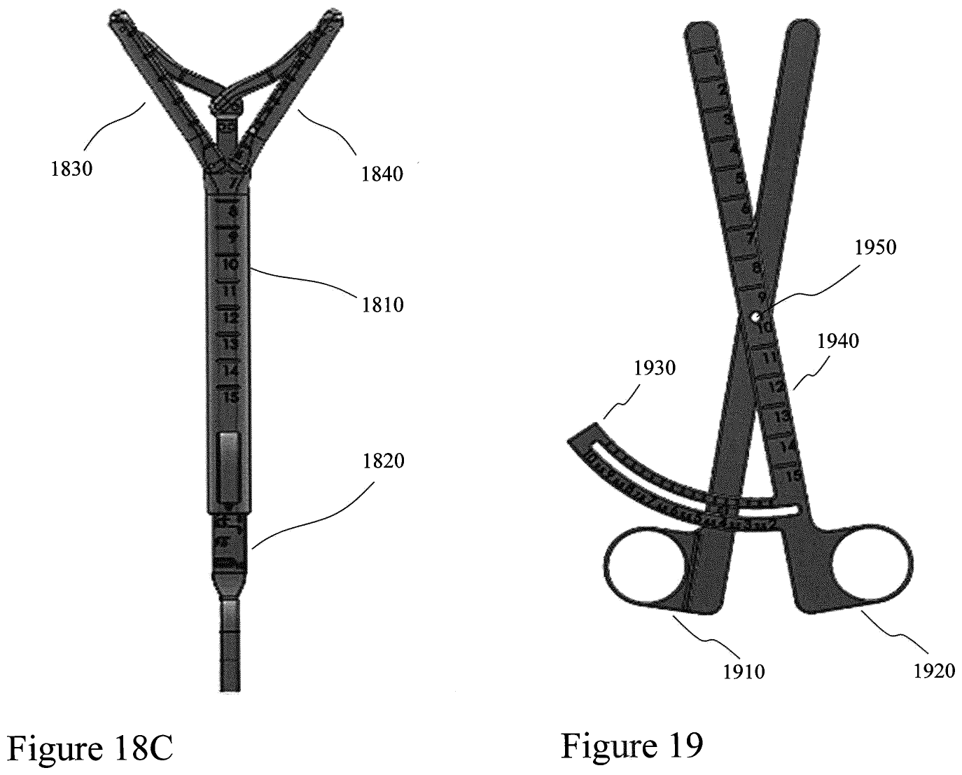

Now referring to FIGS. 18A and 18B there are depicted a POP-Q+ measurement tool (POPQ-Tool) according to an embodiment of the invention in deployed and collapsed states. The inventors referring to this design as a “linkage” design. Accordingly, the POPQ-Tool comprises a central body 1810, a clinician arm 1820 and first and second patient arms 1830 and 1840. The first and second patient arms 1830 and 1840 engaging against the patient's body and moving in dependence upon the clinicians movement of the clinician arm 1820 relative to the central body 1810. The first and second patient arms 1830 and 1840 in FIG. 18A being moved outwards as the clinician arm 1820 is pushed into the central body 1810 by a linkage mechanism where each of the first and second patient arms 1830 and 1840 are attached at a first end (or another point) to the central body 1810 and can rotate about the attachment pivots such that motion of the clinician arm relative to the central body results in the second distal ends of the first and second patient arms moving and changing their lateral separation relative to the longitudinal axis of the central body. Referring to FIG. 18C there is depicted an image of a POP-Q+ tool comprising a variant of the design depicted in FIGS. 18A and 18B respectively. The central body 1810 and first and second patient arms 1830 and 1840 are marked with a common depth scale, from 0 to 15 in this instance, such that with the first and second patient arms 1830 and 1840 in the collapsed state of FIG. 18B a depth measurement of the user's anatomy can be performed. The clinician arm 1820 has one or more additional width scales marked upon it to allow the distance between the tips of the first and second patient arms 1830 and 1840 to be easily read by a clinician performing the POP-Q+ measurements.

It would be evident to one of skill in the art that within other embodiments of the invention other linkage designs may yield the reverse result that it is withdrawal of the clinician arm 1820 from the central body 1810 that opens the tips of the first and second patient arms 1830 and 1840 respectively. Optionally, each of the first and second patient arms 1830 and 1840 may be linked with separate linkage mechanism rather than a common linkage mechanism. Optionally, one or both of the second distal ends of the first and second patient arms 1830 and 1840 may comprise one or more sensors.

Referring to FIG. 19 there is depicted an alternate POPQ-Tool according to an embodiment of the invention. In this instance first and second Arms 1910 and 1920 pivot about a Pivot 1950 in a scissor action. Each of the first and second Arms 1910 and 1920 having an end allowing a clinician or user's finger/thumb to open/close the first and second Arms 1910 and 1920. A Width Scale 1930 is attached to the second Arm 1920 with a marker upon the first Arm 1910 such that as the first and second Arms 1910 and 1920 are moved by the user the width between the outer edges of the first and second Arms 1910 and 1920 at the tips distal to the user's grip can read off from the Width Scale 1930 whilst the POPQ-Tool is inserted into a cavity of the user, e.g. a vagina. The second Arm 1920 also has a Length Scale 1940 upon it such that when the first and second Arms 1910 and 1920 are closed the POPQ-Tool can be inserted and a depth measurement made by reading from the Length Scale 1940. There can be a second variation of the tool where arms are not crossed with each other but parallel.

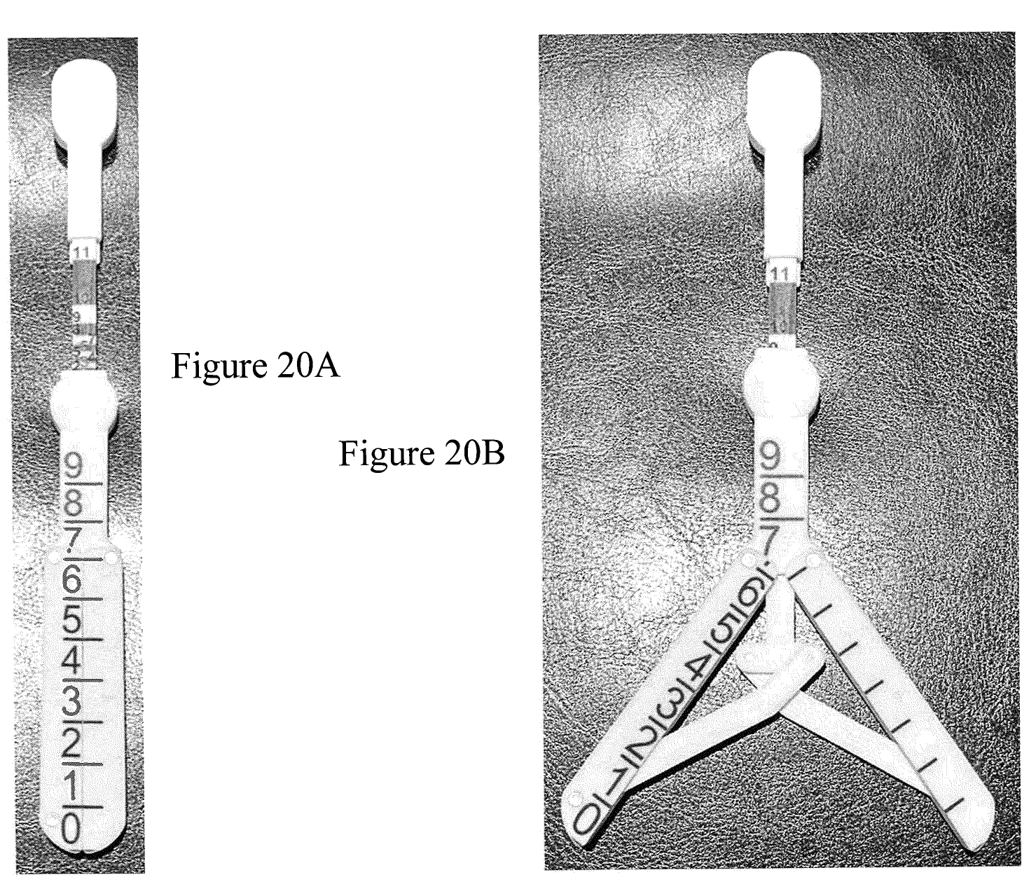

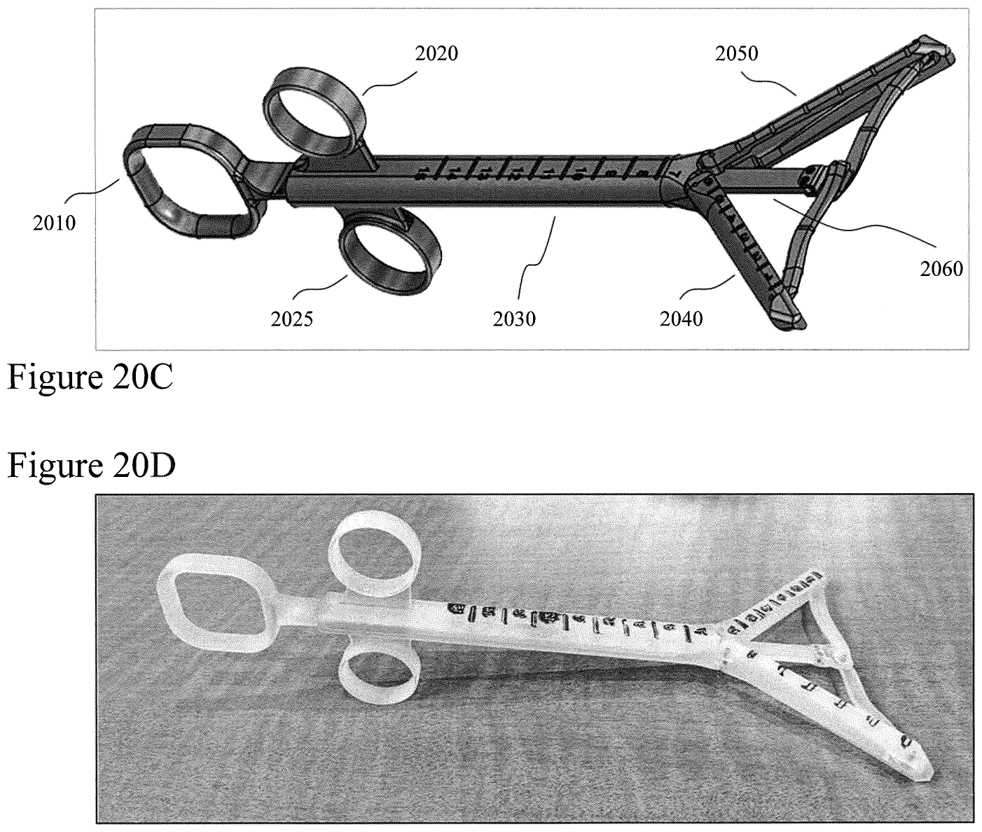

Referring to FIGS. 20A and 20B there are depicted views of a POPQ-Tool according to an embodiment of the invention in collapsed and expanded states respectively. In FIG. 20A, what the inventors refer to as collapsed state, the first and second patient arms and central body are marked with dimensional markings allowing the POPQ-Tool to be employed in performing measurements such as TVL through insertion. The rounded ends of the first and second patient arms providing a low impact insertion means to the patient's vagina. As the clinician arm is pushed into the central body as depicted in FIG. 20B then the POPQ-Tool configures to what the inventors refer to as the expanded state wherein the other visible markings upon the clinician arm at the end of the central body defines the extent to which the first and second patient arms have expanded (extended laterally).

Now referring to FIGS. 20C and 20D there are depicted views of a POPQ-Tool according to an embodiment of the invention in CAD and physical formats. The POPQ-Tool depicted in Figured 20C and 20D operates in a similar manner to the POPQ-Tool depicted in FIGS. 18A-18C and 20B respectively in that it comprises a first Arm 2040 and a second Arm 2050 which are connected to a central Arm 2060 which allows the first and second Arms 2040 and 2050 respectively to be moved between a collapsed state and an expanded state via a user engaging a Handle 2010 on the distal end of the central Arm 2060. A pair of Grips 2020 and 2025 are provided such that a user can hold the POPQ-Tool and extend/retract the central Arm 2060 through the Handle 2010. For example, the user may employ a thumb in conjunction with Handle 2010 and two fingers with the pair of Grips 2020 and 2025 to employ the POPQ-Tool with a single hand. The first and second Arms 2040 and 2050 are pivotably attached to the Body 2030 at one end and to the central Arm 2060 via linkages at their distal ends. In common with the POPQ-Tool depicted in FIGS. 18A-18C and 20B respectively a Length Scale is marked upon the Body 2030 and first and second Arms 2040 and 2050 respectively. A Width Scale reflecting the width of the first and second Arms 2040 and 2050 respectively is provided upon the central Arm 2060 towards the end with the Handle 2010. As depicted with the Handle 2010 fully pushed in the first and second Arms 2040 and 2050 are fully extended and the Width Scale on the central Arm 2060 is not visible.

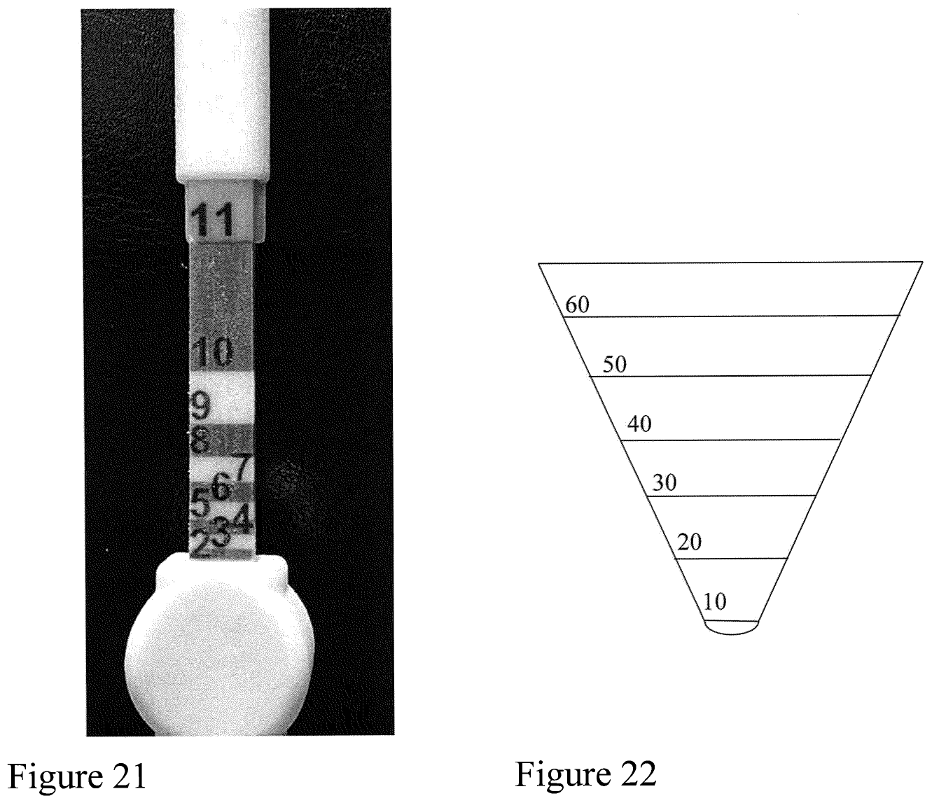

Now referring to FIG. 21 there is depicted a zoomed image of the markings upon the clinician arm of a POPQ-Tool according to an embodiment of the invention depicted the non-linear scale upon the clinician arm which reflects that the distance between the tips of the first and second patient arms does not vary linearly in the design depicted with the position of the clinician arm relative to the central body. Accordingly, the markings on the clinician arm reflect this non-linear relationship. With other mechanisms driving the first and second patient arms apart then the scaling on the clinician arm would be adjusted to reflect this. However, considering FIG. 21 it is evident that relatively little motion of the clinician arm is required to extend the first and second patient arms from 2 cm (0.8″) to 5 cm (2″) for a POP-Q+ measurement tool according to an embodiment of the invention in comparison to the motion required to extend the first and second patient arms from 8 cm (3.15″) to 11 cm (4.3″), a similar 3 cm (1.2″) increase. Optionally, the visual scale may be augmented with a positional encoder and display that measures the position of the clinician arm relative to the central body and performs the appropriate conversion to provide the clinician with the distance between the patient arms.

Referring to FIG. 22 there is depicted a conical measurement scale for a POP-Q+ measurement tool according to an embodiment of the invention for performing an introitus measurement. For example, the POPQ-Tool depicted in FIG. 22 may be attached to the distal end of the clinician arm from the central body of the POPQ-Tool depicted in FIGS. 20A and 20B or it may be a discrete tool with a clinician arm for holding or a u-shaped handle and alike. It would be evident that within other embodiments the measurement scale for the POP-Q+ measurement tool may be non-conical with a profile that varies in lateral dimension(s) along the length of the scale. Within other embodiments of the invention the POP-Q+ tool may have other cross-sectional geometries such as spherical, hemi-spherical, elliptical etc.

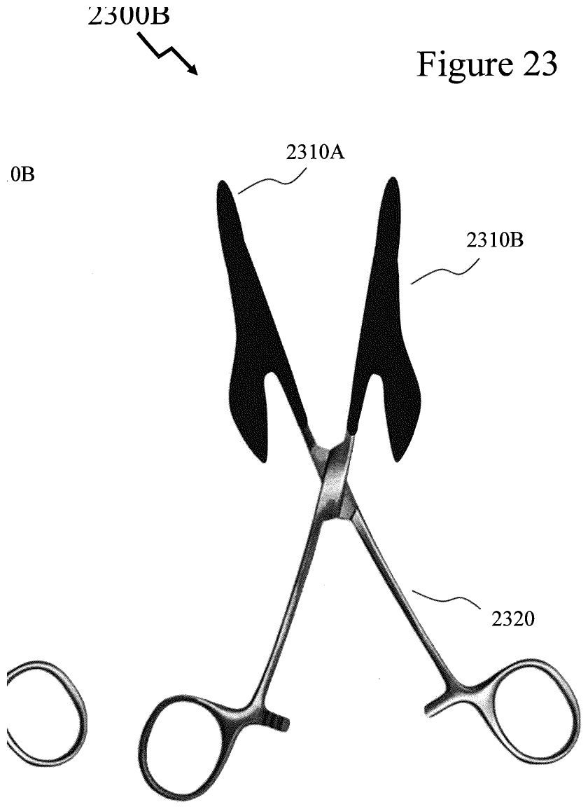

Now referring to FIG. 23 there are depicted first and second Images 2300A and 2300B of demountable attachments to a pair of scissors or forceps to provide a POP-Q+ measurement tool according to an embodiment of the invention. As depicted in first Image 2300A a pair of Forceps 2320 are depicted together with a pair of Demountable Fittings 2310A and 2310B respectively. In second Image 2300B the pair of Demountable Fittings 2310A and 2310B are mounted onto the pair of Forceps 2320 allowing a user to perform the POP-Q+ measurements. The pair of Demountable Fittings 2310A and 2310B may be disposable or they may be reusable, where they are formed from materials compatible with an autoclave or other cleaning/sterilization system. Within another embodiment of the invention the pair of Demountable Fittings 2310A and 2310B may be compatible with curved tipped scissors and/or forceps rather than straight tipped scissors and/or forceps. Within other embodiments of the invention a scale or scales, not depicted in FIG. 23, may be attached to one or both arms of the POPQ-Tool to allow a reading to be made from the other arm as the POPQ-Tool is opened and the pair of Demountable Fittings 2310A and 2310B move laterally. Such scale(s) may be similar to those depicted in FIG. 19 for example.

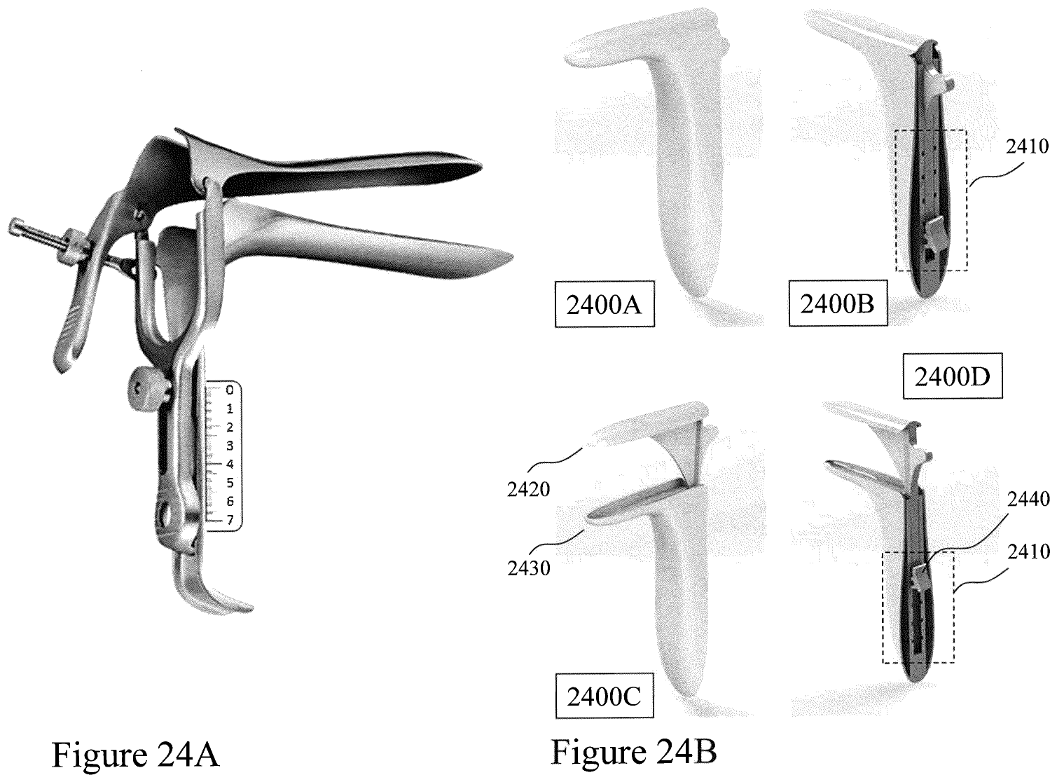

Referring to FIG. 24A there is depicted a POP-Q+ measurement tool according to an embodiment of the invention wherein a scale is attached to a conventional speculum or similar instrument. In contrast in FIG. 24B there are depicted first to fourth Views 2400A to 2400D of a Tool according to an embodiment of the invention. These being front perspective in closed position, rear perspective in closed position, front perspective in open position and rear perspective in open position respectively. In the second and fourth Views 2400B and 2400D respectively the Scale 2410 is depicted allowing a user to establish the opening with of the upper and lower Jaws 2420 and 2430 respectively. A Latch 2440 enables the opening/closing of the upper and lower Jaws 2420 and 2430 respectively of the POPQ-Tool such that the POPQ-Tool can be placed into the open position in situ and latched such that the user can lock the POPQ-Tool and then read the Scale 2410.

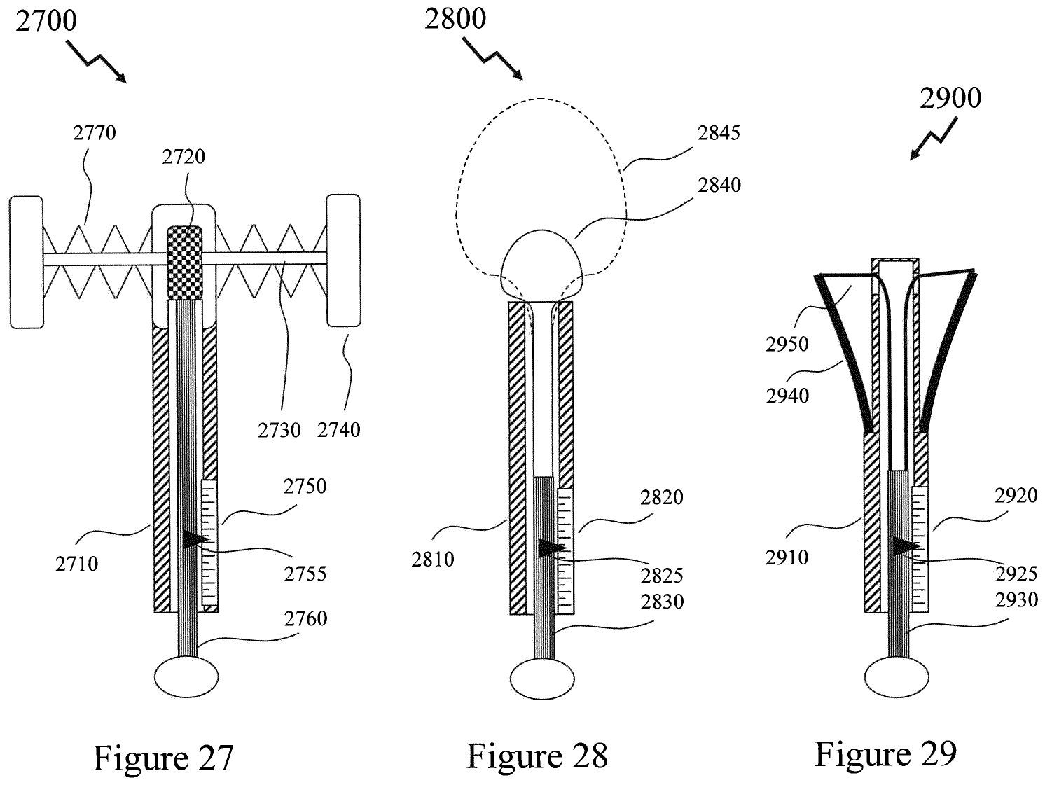

Referring to FIGS. 27 to 29 there are depicted POP-Q_Tools (POPQ-Tools) 2700 to 2900 respectively according to embodiments of the invention. Referring to FIG. 27 the POPQ-Tool 2700 comprises a pair of Pads 2740 at the distal ends of Arms 2730. The other ends of the Arms 2730 being coupled to an Extending Mechanism 2720 that converts motion of the Central Rod 2760 within the Body 2710 into inward or outward motion of the Arms 2730 relative to the Body 2710. A Marker 2755 on the Central Rod 2760 provides a user with a reading of the extension of the Arms 2730 and therein Pads 2740 via Scale 2750. Disposed between each Pad 2740 and the Body 2710 around the Arm 2730 is Retention Means 2770 which acts to pull the Pads 2740 to the Body 2710 such that the Arms 2730 are only moved under the action of the Central Rod 2760.

Within an embodiment of the invention the Central Rod 2760 may rotate to drive the Extending Mechanism 2720 to extend the Arms 2730 directly or via one or more gears which then engage grooves on the Arms 2730 such that the rotator motion of the Central Rod 2760 is converted to linear motion of the Arms 2730. By appropriate gearing increased accuracy can be obtained by gearing down the number of rotations and a larger motion of a marker against a scale to the linear extensions of the arms. Within another embodiment of the invention the Central Rod 2760 may move to push/pull the Arms 2730.

Now referring to FIG. 28 the POPQ-Tool 2800 is depicted comprising a Body 2810, Scale 2820, Marker 2825 and Central Rod 2830. Attached to the end of the Central Rod 2830 is Ribbon 2840 which is a flexible length of a material such that as the Ribbon 2840 is extended out of the bore of the Body 2810 it seeks to return to its original geometry and expands to fit between the walls a cavity it is inserted within. Within an embodiment of the invention a user retracts the Ribbon 2840 into the bore by pulling on the Central Rod 2830, inserts the POPQ-Tool 2800, and releases the Central Rod 2830. The Ribbon 2840 expands automatically into the cavity, e.g. as depicted by Expanded Ribbon 2845 pulling the Central Rod 2830 back. Accordingly, the user can simply read the reading from the Scale 2825. Within another embodiment of the invention the user may push the Central Rod 2830 expanding the Ribbon 2840 until they feel resistance from the Ribbon 2840 against the cavity walls. For example, Ribbon 2840 may be formed from one or more plastics and/or metals.

Referring to FIG. 29 there POPQ-Tool 2900 is depicted. This similarly comprising a Body 2910, Scale 2920, Marker 2925 and Central Rod 2930. The Central Rod 2930 now connecting to an Extending Means 2950 at one end where the other end of the Extending Means 2950 is connected to an elastic deformable Wall 2940 of the POPQ-Tool 2900. Within an embodiment of the invention a user retracts the Extending Means 2950 into the bore by pulling on the Central Rod 2930, inserts the POPQ-Tool 2800, and releases the Central Rod 2930. With the Extending Means 2950 retracted the elastic deformable Wall 2940 is pulled inwards minimizing the lateral extent of the POPQ-Tool 2900. Release of the Central Rod 2930 results in the clastic deformable Wall 2940 seeking to return to its default state wherein it contacts the walls of the cavity. As the clastic deformable Wall 2940 expands the Extending Means 2950 is pulled automatically therein pulling the Central Rod 2930. Accordingly, the user can simply read the reading from the Scale 2925. Within another embodiment of the invention the user may push the Central Rod 2830 extending the Extending Means 2950 and expanding the elastic deformable Wall 2940 until they feel resistance from the elastic deformable Wall 2940 against the cavity walls.

POPQ-Tools according to embodiments of the invention such as those within FIGS. 18A-24 and 27-29 respectively are intended to provide reliable POP-Q examinations for use within custom UTSD design processes. Ideally, a low cost and easy to use tool for performing pelvic examinations it should provided consistent results. Exemplary embodiments may be designed to measure total vaginal length (TVL, the length of the vagina from the posterior fornix to hymen) up to 15 cm (6″) with 0.5 cm (0.2″) or better resolution and widths up to 11 cm (4.3″) with resolution of 0.2 cm (0.08″) or better. A target insertion diameter for the POP-Q tool is less than 2 cm (0.8″).

Whilst embodiments of the invention have been described with respect to scales to provide width measurements based upon a marker moving as the arms are extended etc. removing such linkages of extension/measurement may be beneficial in some instances. Accordingly, disposed within an inner portion of a tip of one arm may be an optical emitter/receiver assembly with a mirror or reflective element upon the inner portion of a tip of the other arm such that through pulses an optical time domain reflectometry measurement of the tip separation can be obtained. Within another embodiment the receiver may be within one arm and the emitter within the other, although this reduces the time of flight to 50% of that of the reflective design.

Within other embodiments of the invention POP-Q tools such as those described with respect to FIG. 27 a mechanical means of distension is employed to move an element radially from the POP-Q tool to engage the cavity wall. However, within other embodiments of the invention the distension may be obtained through fluidic means such that a balloon or balloons are filled with a fluid such that they enlarge wherein the POP-Q tool can be calibrated in terms of lateral width as a function of fluid volume provided to the balloon(s). For example, the fluid may be air, nitrogen, water, a silicone etc. By appropriate design and/or material selection of the balloon low resistance extension can be provided in one direction (i.e. radially from the POP-Q tool) without substantial distension of the balloon in an orthogonal direction (i.e. longitudinally to the POP-Q tool) (see for example U.S. Pat. No. 10,456,323, “Methods and Devices for Fluid Driven Adult Devices”).

Alternatively, the balloon may be within a closed end tube that fits within (or around) another open ended tube forming part of the POP-Q Tool such that as the balloon expands the closed end tube is moved within (or along) the open ended tube such that the closed end tube “floats” and moves. By attaching the balloon to the closed end tube as fluid is removed from the balloon and it shrinks the closed end tube is pulled backwards. Such a concept being depicted within FIG. 30 in first Image 3000A with Fixed Tube 3010 (i.e., open ended tube) within which is Floating Tube 3020 (i.e., closed end tube) with limiting features to prevent Floating Tube 3020 detaching from the Fixed Tube 3010 and therein the POP-Q Tool. The Balloon 3030 is depicted within the Floating Tube 3020 and fluidically connected to the Fixed Tube 3010. Optionally, multiple assemblies my be disposed radially for multiple lateral measurements at one location on the POP-Q tool or at multiple locations along the length of part of the POP-Q tool so that multiple measurements can be obtained concurrently. Such fluidic means allow for automated measurements based upon determining contact of the extending element, e.g. Floating Tube 3020, through a pressure change, back pressure etc.

Alternatively, proximity sensing, time domain reflectometry etc. may be used to determine the extension of the extending element away from the POP-Q Tool body as may electrical techniques such as the extending element providing a variable resistance as it extends to a contact on the POP-Q tool. Alternatively, as depicted in second Image 3000B an elastic element 3040 is disposed from a fixed position on the POP-Q tool to the Floating Tub 3020 such that as the Floating Tube 3020 extends the elastic element 3040 similarly extends moving the Pointer 3050 such that the extension can be read from a scale on the POP-Q tool.

Within another embodiment of the invention a ring is disposed around the tips of a POP-Q Tool which is elastic but has a maximum extension less than that of the arms of the POP-Q tool at their widest. The outer edges of each arm are grooved such that as the POP-Q tool arms open initially the ring expands before it subsequently moves down one groove, through its limiting extension and the profile of the grooves on the arms. This sequence repeats as the arms expand. Once maximum extension is reached the arms are collapsed, the ring contracts but maintains its position on the arms and the tool is removed. The maximum width can then be “read” from the groove to which the ring has moved down to as the arms were extended.

Within other embodiments of the invention the extended arms of the POPQ-Tools such as those depicted in FIGS. 20A to 23 may include one or more sensors to provide additional data relating to providing measurement of the distension of the POPQ-Tool. Optionally, the arms of the POPQ-Tool may be disposable and attached to a caliper or similar instrument to provide a measurement of the distension/opening of the POPQ-Tool. Within embodiments of the invention the accuracy of the measurements provided by the POPQ-Tool through sensors, etc. may be millimeter or sub-millimeter.

Optionally, the POPQ-Tools such as those depicted in FIGS. 20A to 23 may include one or more sensors to provide additional data, e.g. force sensor or another sensor. These may be disposed, for example within the measurement scale of FIG. 22.

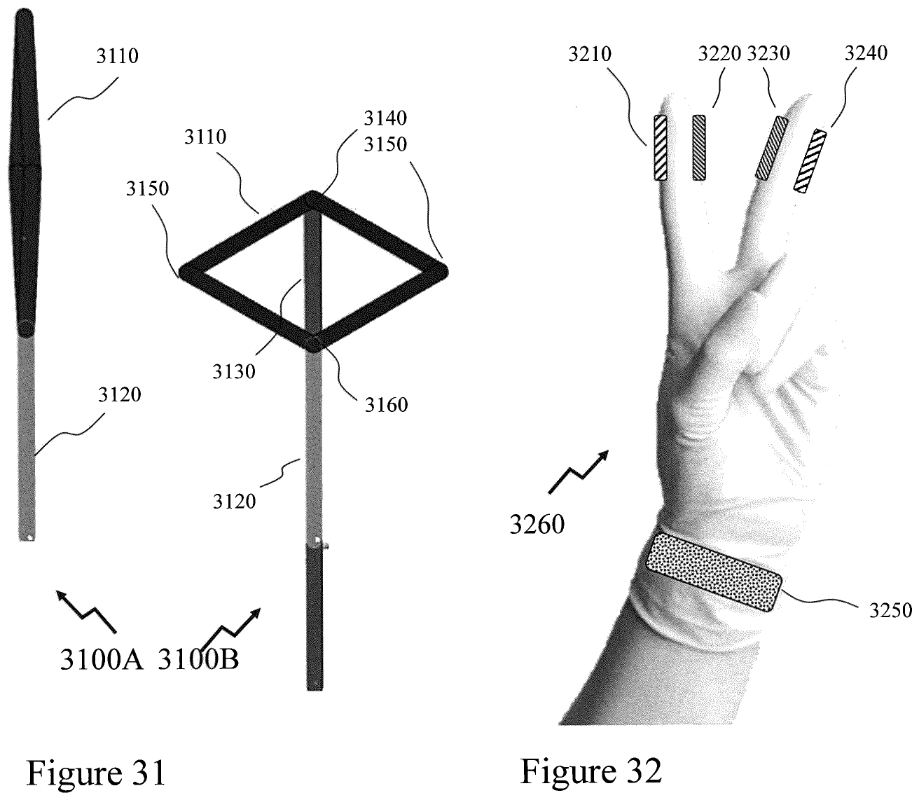

Now referring to FIG. 31 there are depicted first and second Images 3100A and 3100B of an alternate mechanical driving mechanism for a POP-Q+ measurement tool according to an embodiment of the invention. FIG. 31 depicting an alternate mechanical configuration for distending/retracting the Arms 3110 to that depicted in FIGS. 18A-18C and 20B-20D respectively. Each Arm 3110 comprising an upper section attached to Top Pivot 3140 and Lateral Pivot 3150 and a lower section attached to Bottom Pivot 3160 and to the top section at Lateral Pivot 3150. Bottom Pivot 3160 being at an end of Stem 3120. Disposed within the Stem 3120 is Slider 3130 which is connected to the Top Pivot 3140. Accordingly, pulling the Slider 3130 down pulls Top Pivot 3140 down such that the Arms 3110 distend pivoting the upper and lower sections by the Top Pivot 3140, Bottom Pivot 3160 and Lateral Pivots 3150. A scale, not depicted, may be implemented upon the Slider 3130 to provide a visual reading to a user of the POP-Q+ tool.

Referring to FIG. 32 there is depicted a Glove 3260 based POP-Q+ measurement tool for augmenting a manual finger method. Disposed upon and/or within the Glove 3260 are first to fourth Sensors 3210 to 3240 respectively. These may be wired or wirelessly connected to a Controller 3250 which is then wired or wirelessly connected to an external system for logging the measurements. Within an embodiment of the invention the second and third Sensors 3230 and 3240 may an optical emitter/optical receiver such that through pulses an optical time domain reflectometry measurement of the tip separation can be obtained. Within an embodiment of the invention the first and fourth Sensors 3210 and 3240 may be pressure and/or force sensors such that pressure/force measurements can be made or monitored till a predetermined pressure and/or force is achieved at which point the distance between the two fingers is measured.

Within another embodiment of the invention the finger(s) of the Glove 3260 may comprise other sensors such as temperature, oxygenation sensors, etc. Within another embodiment of the invention the finger(s) of the Glove 3260 may include accelerometers for 3D orientation and positioning information to be obtained.

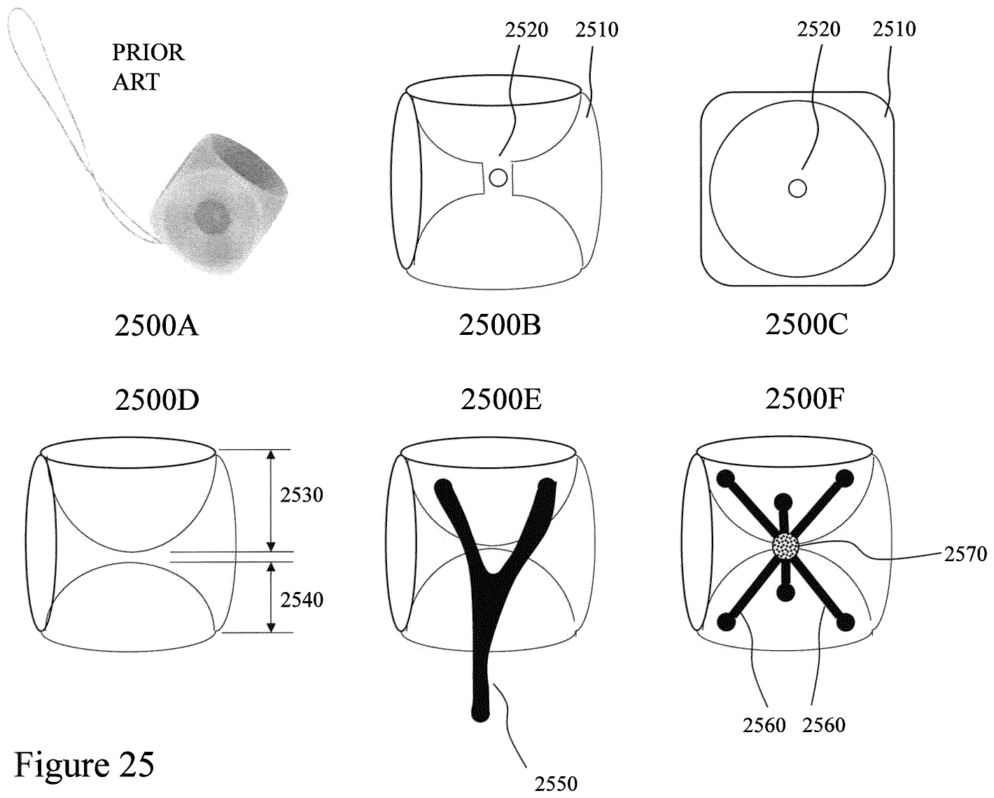

Now referring to FIG. 25 there are depicted first to fifth non-perforated cube pessaries (NPCPs) 2500B to 2500F respectively according to embodiments of the invention relative to a prior art NPCP 2500A. Referring to first NPCP 2500B there is depicted a NPCP 2510 according to an embodiment of the invention for use with a pump via a tube (both of which are not depicted for clarity) wherein the tube is inserted into the NPCP 2510 via the Opening 2520. Accordingly, the NPCP 2510 can be inflated with a fluid after insertion (e.g. air or water) or the NPCP 2510 may be deflated for removal. The Opening 2520 may, for example, be part of a simple non-return valve (NRV) or check valve wherein an element within the NRV or check valve is pushed open by the insertion of the tube and closes when the tube is removed. Second NPCP 2500C depicts the Opening 2500 within a face of NPCP 2500B rather than an edge as depicted with first NPCP 2500A.

Third NPCP 2500D does not include an opening but is designed asymmetrical rather than symmetrical as with prior art NPCP 2500A. Accordingly, a portion of first dimension 2530 is larger than another portion of second dimension 2540. Third NPCP 2500D is removed using what is known as a “pinch-and-pull” methodology wherein the larger portion of first dimension 2530 can be reached easier by the clinician or user when seeking to “pinch” the NPCP and remove it.

In contrast fourth NPCP 2500E has a Pull Tab 2550 attached at two points upon the body of the fourth NPCP 2500E such that when the Pull Tab 2550 is pulled with sufficient force it results in the fourth NPCP 2500E being pulled releasing suction between the fourth NPCP 2500E and the user's body. Within other embodiments of the invention the Pull Tab 2550 may be attached to one point, three points or more. Alternatively, the Pull Tab 2550 is attached to opposite sides of the fourth NPCP 2500E such that pulling upon the Pull Tab 2550 results in distortion of the fourth NPCP 2500E geometry releasing suction. For example, the distortion is a pulling in of the portion fourth NPCP 2500E to which the Pull Tab 2550 is attached or a pulling in of another portion of the fourth NPCP 2500E distal to the end to which the Pull Tab 2550 is attached.

Alternatively, as depicted with fifth NPCP 2500F a Twist Tab 2570 is attached multiple points upon the fifth NPCP 2500F wherein the Twist Tab 2570 upon being twisted shortens upon each Arm 2560 that runs from the Twist Tab 2570 to an attachment point on the fifth NPCP 2550F and applies a loading to the fifth NPCP 2500F such that the resulting distortion of the fifth NPCP 2500F is a pulling in of the portion fifth NPCP 2500F to which the Twist Tab 2570 is attached or a pulling in of another portion of the fifth NPCP 2500F distal to the end to which the Twist Tab 2570 is attached.

Within other embodiments of the invention an NPCP may be formed from an elastic material or materials wherein the NPCP is initially compressed into a portion of an applicator before being released from the applicator wherein the NPCP expands against the user's body, potentially to its maximum original extent although more likely to less than its fullest extent. For example, the applicator may include a plunger style assembly to push the NPCP out of the applicator when inserted into the user or patient. In order to enable appropriate compression of the NPCP into the cavity or other portion of the applicator it is stored in the NPCP may be designed to fold or collapse along defined regions. Optionally, these defined regions may be formed from one or more elastomeric materials with the other regions formed from non-elastomeric materials or the entire NPCP may be formed from elastomeric materials.

Alternatively, within other embodiments of the invention a collapsible NPCP may incorporate fold lines in the body of the NPCP to make its use with an applicator or its insertion/removal without an applicator easier for the user or patient. These may, for example, be mechanical flexures which act as hinges with a continuous sheet of material thinner at the “hinge” than either side.

Alternatively, within other embodiments of the invention portions of the NPCP may be formed from one or more auxetic materials or auxetic structures. An auxetic material or auxetic structure being one that has negative Poisson's ratio whereby when stretched they become thicker perpendicular to the applied force. Accordingly, an auxetic material or auxetic structure based NPCP may be inserted, subject to tension whereby it expands and deployed. Subsequently application of compression to the NPCP would result in its collapse and cased removal.

Within the preceding description with respect to third to fifth NPCPs, applicators flexures, etc. these have been described and depicted with respect to non-perforated cube pessary. However, it would be evident that these may also be applied to perforated cube pessaries as well.

It would be evident that auxetic materials and auxetic structures whilst described above with respect to applicators may be construed as applying to non-perforated cube pessaries it would be evident that they may be employed within other embodiments of the invention in conjunction with perforated cube pessaries or other pessaries where their behavioral characteristics are desirable for insertion/removal or adjustment for example.

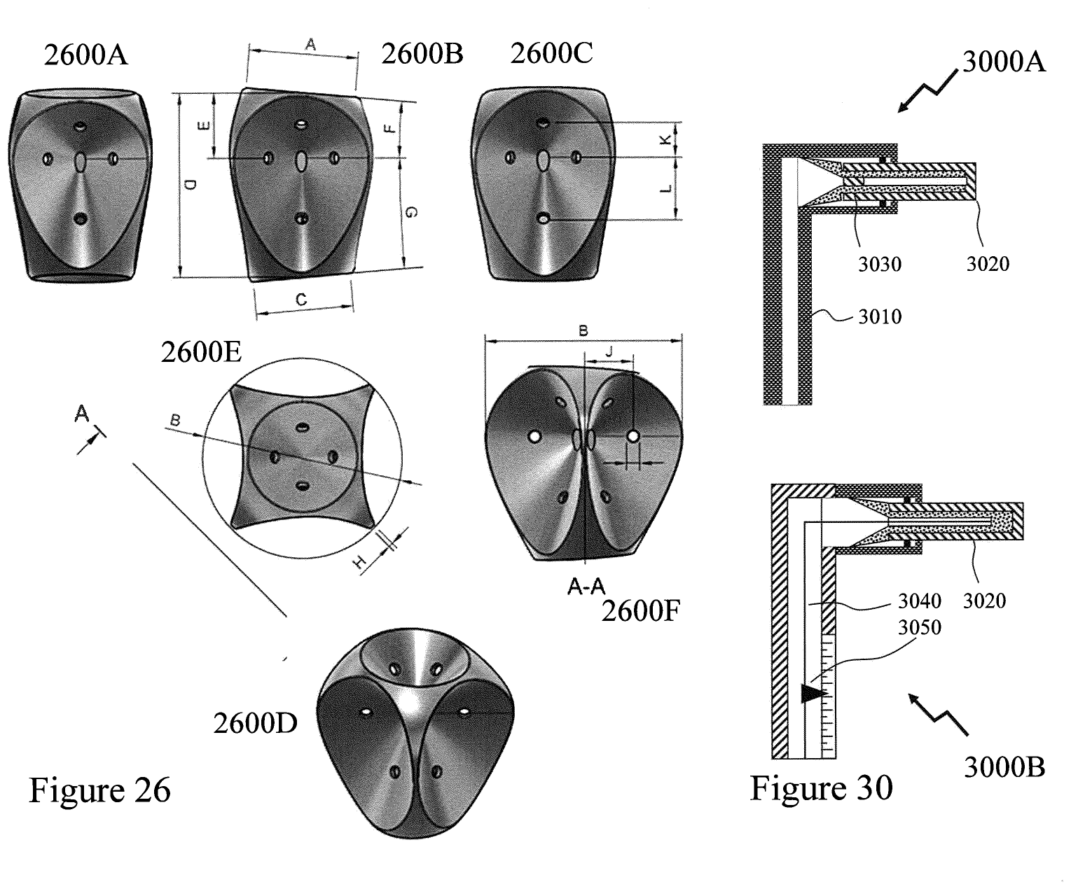

Now referring to FIG. 26 there are depicted first to sixth perspective views 2600A to 2600F for perforated cube pessaries (PCPs) according to embodiments of the invention. Also depicted within these are dimensions as outlined below that define the PCP geometry. As depicted in FIG. 26 first to sixth perspective views 2600A to 2600F are respectively, a left elevation, front view, right elevation, perspective, plan and view along plane A-A.

The depicted dimensions being:
A—top circle diameter of upper surface of PCP;
B—maximum lateral dimension or diameter of the PCP;
C—bottom circle diameter of lower surface of PCP;
D—overall length of PCP along longitudinal axis;
E—upper axial length of PCP;
F—angle of top surface relative to an axis perpendicular to the longitudinal axis;
G—angle of bottom surface relative to an axis perpendicular to the longitudinal axis;
H—thickness of “edge” of PCP;
I—diameter of perforations within PCP;
J—distance of side perforations away from longitudinal axis;
K—distance of upper perforations away from longitudinal axis; and
L—distance of lower perforations away from longitudinal axis.

Whilst the PCP depicted in FIG. 26 has circular upper and lower surfaces and overall circularly symmetric design it would be evident that within other embodiments of the invention the PCP may be defined with an elliptical structure such that the upper and lower surfaces are elliptical. Further, whilst all perforations are depicted as of the same size it would be evident that within other embodiments different dimensions for different holes disposed along the longitudinal axis may be employed and that the number, position of the perforations may change without departing from the scope of the invention. The perforations may also be referred to as discharge holes.

The USTD may be provided in a range of physical sizes such that, for example, the length of an inserted actuated member (e.g. for vaginal insertion) may be 50 mm, 65 mm, 75 mm, 100 mm, 125 mm, or 150 mm for example (2″, 2.5″, 3″, 4″, 5″, or 6″) or other values for this dimension and its lateral dimensions may be, for example, 40 mm, 50 mm, 65 mm, 75 mm or 100 m (1.6″, 2″, 2.5″, 3″, or 4″) or other values for this dimension. The construction of a USTD may employ one or more central scaffolds which provides rigidity or structure to the required portions of the USTD which may be surrounded by a shell and then a casing. Whilst the casing and shell may be transparent or semi-transparent over portions or all of the USTD, cup, plug, cushion etc. it is common for the USTD, cup, plug, cushion etc. to be opaque. An outer casing may be coloured based upon skin colour tones based upon ethnicity or personal preference, e.g. light, dark, etc. as well as single colour, binary colour, multiple colour etc. According to the complexity acceptable then the outer casing may be formed from a variety of colours and/or be patterned for a specific design. Typically, such colours will be part of a silicone or other elastomer employed in forming the casing although in other embodiments of the invention the casing may be coloured once formed and a protective fluid proof, non-toxic, non-abrasive coating formed atop these applied colours. Such instances of applied colours may include metallic lacquers, particulate lacquers for “sparkle”, etc.

Beneficially, medical grade silicone is clear thereby removing the requirement for any additional coating (e.g. food grade urethane) in conjunction with pigmented silicones. Accordingly, an USTD may with medical grade silicone be clear and formed from an initial sticky soft silicone, e.g. 20 durometers, with a micro-layer (spray coated for example) of high durometer medical grade silicone, for example 70-90 durometer, to create “slippery” surface and avoid silky smooth surface that typically requires use of urethane coating.

Typically, the casing for the USTD will be formed from a non-toxic, hypoallergenic silicone to provide a safe smooth surface although some regions of the USTD may be coated, textured and/or finished with a variation from that of the remainder of the casing in order to enhance or promote retention of the USTD against the user's skin or clothing. Typically, the outer surface of the casing will be formed to provide low friction as well as resistance to lubricants, spermicides, and other chemicals that may or may not be employed by the user.

Embodiments of the invention with respect to the USTD such as described within the embodiments of the invention supra may employ a “sticky” surface for a predetermined portion of the outer surface for engaging a recipient's body (e.g. being formed from a low durometer silicone for example) so that the surface is designed to “stick” to skin, so it stays in place or has higher resistance to motion. This “sticky” surface may be mirror surface, matt or textured for grip. Examples of materials may be those with durometer ideal Shore A10 or lower, Shore A5 or lower, or Shore A1. In some embodiments of the invention a region or regions of the casing may be formed from a gel such as the Ecoflex™ platinum catalyzed silicones for example certified to ISI 10993-10 for skin irritation/sensitization and having, for example, Shore 00-50 hardness (below the Shore A scale), Shore 00-30 hardness, Shore 00-20 hardness, or Shore 00-10 hardness. In embodiments of the invention the casing around the shell may act like a thin sheet (<<1 mm thick), like a fabric or material, like a sheet (˜1 mm), a thick sheet (>1 mm). Optionally, the lower surface of the casing designed for placement against a user's groin/stomach may be sticky and when washed recover this stickiness in its entirety or in different regions or areas.

Optionally, the outer surface which contact the user may be smooth with low friction to human skin, smooth with minimal friction to human skin, smooth with moderate friction to human skin, smooth with high friction to human skin in its entirety or in different regions or areas. Alternatively, the surface may be smooth, textured, and/or rough and have low friction, negligible friction, moderate friction, and/or high friction in its entirety or in different regions or areas. Optionally, the surface may be textured with low friction to human skin, textured with minimal friction to human skin, textured with moderate friction to human skin, or textured with high friction to human skin in its entirety or in different regions. Optionally, the surface of the casing in its entirety or in different regions or areas may be used in conjunction with disposable sheets that provide adhesion and/or friction in predetermined levels.

Within embodiments of the invention the casing, for example formed from silicone, is the only material surrounding the casing and the surface profile is derived from applying the casing to the contoured surface of the shell. In other embodiments of the invention the surface profile is derived from multiple applications of a single material forming the casing. In other embodiments of the invention an additional material or materials are disposed between the shell and the casing. This, may for example, be a preform formed from the same material as the casing such that the casing is applied as a single or multiple dip coating for example, a preform formed from another silicone of different characteristics to the casing, a preform formed from a plastic, a preform formed from a low density foam, from a medium density foam, or a high density foam. Alternatively, a combination of materials may be employed such as two or more plastics, two or more foams, a foam and a plastic, a foam and a silicone, a form and metal. The materials may be layered, inserted, embedded, etc. without departing from the scope of the invention. However, a characteristic of these materials is the transmission of vibratory motion arising from the active elements within the USTD according to embodiments of the invention. Within passive embodiments this characteristic of material selection is removed.

Optionally, the USTDs are formed through either one or more additive manufacturing (AM) steps and/or one or more non-additive manufacturing (NAM) steps.

The foregoing disclosure of the exemplary embodiments of the present invention has been presented for purposes of illustration and description. It is not intended to be exhaustive or to limit the invention to the precise forms disclosed. Many variations and modifications of the embodiments described herein will be apparent to one of or §§ dinary skill in the art in light of the above disclosure. The scope of the invention is to be defined only by the claims appended hereto, and by their equivalents.

Further, in describing representative embodiments of the present invention, the specification may have presented the method and/or process of the present invention as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present invention should not be limited to the performance of their steps in the order written, and one skilled in the art can readily appreciate that the sequences may be varied and still remain within the spirit and scope of the present invention.

What is claimed is:

1-16. (canceled)

17. A device comprising:
an outer ring having a predetermined geometry;
an upper opening having a distal end proximate to the outer ring;
a plurality of ribs, wherein each rib of the plurality of ribs has a first end at a predetermined position on the upper opening and a second end at a predetermined position on the outer ring.

18. The device according to claim 17, wherein: the upper opening further comprises a hollow central shaft.

19. The device according to claim 18, further comprising:
one or more pull-tabs, wherein each of the one or more pull-tabs has: a first pull-tab end, the first pull-tab end attached to a further predetermined position on the outer ring and a second pull-tab distal end;
and wherein the at least one pull-tab extends from the first pull-tab end through an opening in a sidewall of the hollow central shaft and through the second distal end of the hollow central shaft.

20. The device according to claim 17, further comprising one or more of:
a pull-tab having a first distal end attached at a predetermined position on the outer ring;
one or more membranes, the one or more membranes being disposed within the outer ring, each of the one or more membranes having a plurality of openings; and
a plurality of notches formed into a lower surface of the outer ring.

21. The device according to claim 18, further comprising:
a non-return valve coupled to one or more of: the outer ring and the upper opening; and
wherein the one or more of the outer ring and the upper opening, are hollow and fillable through the non-return valve.

22. The device according to claim 17, further comprising one or more of:
a knob disposed at a predetermined position on the exterior of the outer ring and a pull-tab having a first distal end attached at another predetermined position on the outer ring; and
a membrane disposed within the outer ring having a plurality of openings through it; and
a knob disposed at a predetermined position on the exterior of the outer ring and a pull-tab having a first distal end attached at another predetermined position on the outer ring aligned with the knob.

23. The device according to claim 17, further comprising one or more of:
a plurality of pull-tabs, each pull-tab of the plurality of pull-tabs having a first end attached at a predetermined position on the outer ring and a second distal end attached to the second distal ends of the other pull-tabs of the plurality of pull-tabs;
a plurality of pull-tabs, each pull-tab of the plurality of pull-tabs having a first end attached at a predetermined position on the outer ring and a second distal end attached to the second distal ends of the other pull-tabs of the plurality of pull-tabs wherein the predetermined position of a first portion of the plurality of pull-tabs is located on the outer ring and forms an inner periphery of the outer ring and the predetermined position of a second portion of the plurality of pull-tabs is located on the outer ring and forms an outer periphery of the outer ring;
a membrane disposed within the outer ring having a plurality of openings through it and a plurality of pull-tabs, each pull-tab of the plurality of pull-tabs having a first end attached at a predetermined position on the outer ring and a second distal end attached to the second distal ends of the other pull-tabs of the plurality of pull-tabs; and
a membrane disposed within the outer ring having a plurality of openings through it and a plurality of pull-tabs, each pull-tab of the plurality of pull-tabs having a first end attached at a predetermined position on the outer ring and a second distal end attached to the second distal ends of the other pull-tabs of the plurality of pull-tabs where the predetermined position of a first portion of the plurality of pull-tabs have is located on the outer ring and forms an inner periphery of the outer ring and the predetermined position of a second portion of the plurality of pull-tabs have is located on the outer ring and forms an outer periphery of the outer ring.

24. A measurement device comprising:
a central body having a longitudinal axis;
a clinician arm having a first end disposed within the central body and a second distal end disposed external to the central body;
a first patient arm having a first end and a second distal end, wherein the first patient arm is disposed at a predetermined position with respect to the central body on a first side of the longitudinal axis, connected at a predetermined position to the central body via a first pivot, and connected via a first linkage mechanism to the clinician arm;
a second patient arm having a first end and a second distal end, wherein the second patient arm is disposed at a predetermined position with respect to the central body on a second side of the longitudinal axis, connected at a predetermined position to the central body via a second pivot, and connected via a second linkage mechanism to the clinician arm; and
wherein movement of the clinician arm relative to the central body results in translation of the second distal ends of the first patient arm and the second patient arm laterally with respect to the longitudinal axis.

25. The device according to claim 24, further comprising one or more measurement scales wherein the one or more measurement scales comprise one or more of:
a first scale disposed upon the clinician arm to provide a user of the device with a visual indication of the lateral separation between the second distal ends of the first patient arm and the second patient arm;
wherein the first scale is selected from the group comprising: linear scale, a non-linear scale, a varying scale which varies in a predetermined manner such that its diameter increases along its length from the second distal end of the clinician arm towards the first end of the clinician arm; and
a second scale disposed upon the clinician arm to provide a user of the device with a visual indication of the diameter of the measurement scale at its current location when inserted into a vagina of a user.

26. The measurement device according to claim 24, further comprising one or more sensors, each sensor of the one or more sensors disposed within a second distal end of one of the first patient arm and the second patient arm.

27. The measurement device according to claim 26, wherein the one or more sensors comprise one or more of: distance sensor, optical sensors, force sensors, pressure sensors, biometric sensors, temperature, oxygenation, accelerometers.

28. A measurement device comprising:
a first sensor disposed upon a first side of a measurement tool;
a second sensor disposed upon a second side of a measurement tool; and
a controller connected to the first and second sensors by a connection mechanism, the controller configured to measure a value relating to a separation of the first and second sensors;
wherein the measurement tool is selected from the group comprising: a pelvic organ prolapse quantification (POP-Q) tool, gloves, fingers, scissors, and forceps.

29. The measurement device according to claim 28, wherein the first and second sensors are two or more of: distance sensors, optical sensors, force sensors, pressure sensors, biometric sensors, temperature, oxygenation, accelerometers and the value measured by the controller is a distance measurement.

30. The measurement device according to claim 28, further comprising:
a third and fourth sensor connected to the controller by a second connection mechanism, the third and fourth sensors being one or more of pressure and force sensors; and
wherein the controller is further configured to, upon measuring a pressure or force from the third and fourth sensors that meets a predetermined threshold, automatically measure a distance between the first and second sensors.

31. The measurement device according to claim 28, wherein the connection mechanism is one or more of: a wireless or wired connection mechanism.
